(19) United States
(12) Patent Application Publication
(10) Pub. No.: US 2014/0004097 A1
(43) Pub. Date: Jan. 2, 2014

(54) METHOD OF PRODUCING RECOMBINANT IDURONATE-2-SULFATASE

(71) Applicant: SHIRE HUMAN GENETIC THERAPIES, INC., LEXINGTON, MA (US)

(72) Inventors: Chun Zhang, Lexington, MA (US); Ferenc Boldog, Lexington, MA (US)

(21) Appl. No.: 13/829,811

(22) Filed: Mar. 14, 2013

Related U.S. Application Data
(60) Provisional application No. 61/666,712, filed on Jun. 29, 2012.

(51) Int. Cl. C12N 9/16

(52) U.S. Cl. CPC C12N 9/16
USPC 424/94.6; 435/196

(57) ABSTRACT

The present invention provides, among other things, methods and compositions for large-scale production of recombinant I2S protein using suspension culture of mammalian cells in serum-free medium. In particular, the present invention uses mammalian cells co-express a recombinant I2S protein and a formylglycine generating enzyme (FGE).

### CROSS REFERENCE TO RELATED APPLICATIONS

This application claims benefit under 35 USC §119(e) of U.S. Provisional Patent Application Ser. No. 61/666,712, filed Jun. 29, 2012, which application is hereby incorporated by reference in its entirety.

### SEQUENCE LISTING

The present specification makes reference to a Sequence Listing submitted in electronic form as an ASCII .txt file named “2006685-0276 SEQ LIST” on Mar. 14, 2013. The .txt file was generated on Mar. 5, 2013 and is 21 KB in size. The entire contents of the Sequence Listing are herein incorporated by reference.

### BACKGROUND

Mucopolysaccharidosis type II (MPS II, Hunter syndrome) is an X-chromosome-linked recessive lysosomal storage disorder that results from a deficiency in the enzyme iduronate-2-sulfatase (I2S). I2S cleaves the terminal 2-O-sulfate moieties from the glycosaminoglycans (GAG) dermatan sulfate and heparan sulfate. Due to the missing or defective I2S enzyme in patients with Hunter syndrome, GAG progressively accumulate in the lysosomes of a variety of cell types, leading to cellular engorgement, organomegaly, tissue destruction, and organ system dysfunction.

Generally, physical manifestations for people with Hunter syndrome include both somatic and neuronal symptoms. For example, in some cases of Hunter syndrome, central nervous system involvement leads to developmental delays and nervous system problems. While the non-neuronal symptoms of Hunter Syndrome are generally absent at birth, over time the progressive accumulation of GAG in the cells of the body can have a dramatic impact on the peripheral tissues of the body. GAG accumulation in the peripheral tissue leads to a distinctive coarseness in the facial features of a patient and is responsible for the prominent forehead, flattened bridge and enlarged tongue, the defining hallmarks of a Hunter patient. Similarly, the accumulation of GAG can adversely affect the organ systems of the body. Manifesting initially as a thickening of the wall of the heart, lungs and airways, and abnormal enlargement of the liver, spleen and kidneys, these profound changes can ultimately lead to widespread catastrophic organ failure. As a result, Hunter syndrome is always severe, progressive, and life-limiting.

Enzyme replacement therapy (ERT) is an approved therapy for treating Hunter syndrome (MPS II), which involves administering exogenous replacement I2S enzyme to patients with Hunter syndrome.

### SUMMARY OF THE INVENTION

The present invention provides, among other things, an improved method for large scale production of recombinant I2S enzyme to facilitate effective treatment of Hunter syndrome. Prior to the present invention, roller bottle adherent culture system using serum-containing medium has been successfully developed to produce recombinant I2S at large scale. The inventors of the present application however developed a system that can effectively cultivate mammalian cells co-expressing I2S and formylglycine generating enzyme (FGE) in suspension in a large scale vessel using animal-component free, chemically-defined medium to efficiently produce a large quantity of recombinant I2S enzyme. Unexpectedly, a recombinant I2S enzyme produced using the animal-free suspension culturing system also has significantly improved enzymatic activity because the recombinant I2S produced in this fashion has an unusually high level of C_{α}-formylglycine (FGly) (e.g., above 70% and up to 100%), which is required for the activity of I2S. In addition, the recombinant I2S enzyme produced according to the present invention has distinct characteristics such as sialic acid content and glycan map, which may improve bioavailability of the recombinant I2S protein. Moreover, the animal free culture system simplifies the downstream purification process and reduces or eliminates serum-originated contaminants such as fetuin. Thus, the present invention provides a large scale production system that is more efficient, cost-effective, reproducible, safer and produces more potent recombinant I2S.

Thus, in one aspect, the present invention provides a method for large-scale production of recombinant iduronate-2-sulfatase (I2S) protein in mammalian cells by culturing mammalian cells co-expressing a recombinant I2S protein and a formylglycine generating enzyme (FGE) in suspension in a large-scale culture vessel containing medium lacking serum. In some embodiments, the culturing step involves a perfusion process.

In another aspect, the present invention provides a method for large-scale production of recombinant iduronate-2-sulfatase (I2S) protein in mammalian cells, comprising culturing mammalian cells co-expressing a recombinant I2S protein and a formylglycine generating enzyme (FGE) in a large-scale culture vessel containing medium lacking serum under conditions such that the cells, on average, produce the recombinant I2S protein at a specific productivity rate of great than about 15 picogram/cell/day and further wherein the produced recombinant I2S protein, on average, comprises at least about 60% conversion of the cysteine residue corresponding to Cys59 of human I2S protein to C_{α}-formylglycine. In some embodiments, the culturing step involves a perfusion process.

In some embodiments, the perfusion process has a perfusion rate ranging from about 0.5-2 volume of fresh medium/working volume of reactor/day (VVD) (e.g., about 0.5-1.5 VVD, about 0.75-1.5 VVD, about 0.75-1.25 VVD, about 1.0-2.0 VVD, about 1.0-1.9 VVD, about 1.0-1.8 VVD, about 1.0-1.7 VVD, about 1.0-1.6 VVD, about 1.0-1.5 VVD, about 1.0-1.4 VVD, about 1.0-1.3 VVD, about 1.0-1.2 VVD, about 1.0-1.1 VVD). In some embodiments, the perfusion process has a perfusion rate of about 0.5, 0.55, 0.6, 0.65, 0.7, 0.75, 0.8, 0.85, 0.9, 0.95, 1.0, 1.05, 1.10, 1.15, 1.2, 1.25, 1.3, 1.35, 1.4, 1.45, 1.5, 1.55, 1.6, 1.65, 1.7, 1.75, 1.8, 1.85, 1.9, 1.95, or 2.0 VVD.

In some embodiments, the perfusion process has a cell specific perfusion rate ranging from about 0.05-5 nanoliter per cell per day (nL/cell/day) (e.g., about 0.05-4 mL/cell/day, about 0.05-3 mL/cell/day, about 0.05-2 mL/cell/day, about 0.05-1 mL/cell/day, about 0.1-5 mL/cell/day, about 0.1-4 mL/cell/day, about 0.1-3 mL/cell/day, about 0.1-2 mL/cell/day, about 0.1-1 mL/cell/day, about 0.15-5 mL/cell/day, about 0.15-4 mL/cell/day, about 0.15-3 mL/cell/day, about 0.15-2 mL/cell/day, about 0.15-1 mL/cell/day, about 0.2-5 mL/cell/day, about 0.2-4 mL/cell/day, about 0.2-3 mL/cell/day, about 0.2-2 mL/cell/day, about 0.2-1 mL/cell/day, about 0.25-5 mL/cell/day, about 0.25-4 mL/cell/day, about 0.25-3 mL/cell/day, about 0.25-2 mL/cell/day, about 0.25-1 mL/cell/day, about 0.3-5 mL/cell/day, about 0.3-4 mL/cell/day, about 0.3-3 mL/cell/day, about 0.3-2 mL/cell/day, about 0.3-1 mL/cell/day, about 0.35-5 mL/cell/day, about 0.35-4 mL/cell/day, about 0.35-3 mL/cell/day, about 0.35-2 mL/cell/day, about 0.35-1 mL/cell/day, about 0.4-5 mL/cell/day, about 0.4-4 mL/cell/day, about 0.4-3 mL/cell/day, about 0.4-2 mL/cell/day, about 0.4-1 mL/cell/day, about 0.45-5 mL/cell/day, about 0.45-4 mL/cell/day, about 0.45-3 mL/cell/day, about 0.45-2 mL/cell/day, about 0.45-1 mL/cell/day, about 0.5-5 mL/cell/day, about 0.5-4 mL/cell/day, about 0.5-3 mL/cell/day, about 0.5-2 mL/cell/day, about 0.5-1 mL/cell/day). In some embodiments, the perfusion process has a cell specific perfusion rate of about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mL/cell/day.

In some embodiments, the cells cultivated according to the present invention, on average, produce the recombinant I2S protein at a specific productivity rate of great than about 20 picogram/cell/day (e.g., greater than about 25, 30, 35, 40, 45, 50, 55, 60, 65, 70, 75, 80, 85, 90, 95, or 100 picogram/cell/day). In some embodiments, the cells cultivated according to the present invention produce the recombinant I2S protein at an average harvest titer of at least 6 mg per liter per day (mg/L/day) (e.g., at least 8, 10, 12, 14, 16, 18, 20, 25, 30, 35, 40, 45, 50, 55, 60, 65, 70, 75, 80, 85, 90, 95, 100, 150, 200, 250, 300, 350, 400, 450, or 500 mg/L/day, or more).

In some embodiments, the produced recombinant I2S protein according to a method of the invention comprises at least about 70% (e.g., at least about 75%, 80%, 85%, 90%, 95%, 96%, 97%, 98%, 99%, 100%) conversion of the cysteine residue corresponding to Cys59 of human I2S protein to C_{α}-formylglycine (FGly).

In some embodiments, mammalian cells suitable for the present invention are human cells. In some embodiments, mammalian cells suitable for the present invention are CHO cells.

In some embodiments, a large-scale culture vessel suitable for the present invention is a bioreactor. In some embodiments, a suitable bioreactor is at a scale of or greater than 10 L, 200 L, 500 L, 1000 L, 1500 L, 2000 L, 2500 L, 3000 L.

In some embodiments, a medium suitable for the present invention lacks animal-derived components. In some embodiments, a suitable medium is chemically-defined medium. In some embodiments, a suitable medium is protein free.

In some embodiments, a medium suitable for the present invention contains at least one redox-modulator. In some embodiments, a redox-modulator suitable for the present invention is selected from the group consisting of glutathione, glucose-6-phosphate, carnosine, carnosol, sulforaphane, tocopherol, ascorbate, dehydroascorbate, selenium, 2-mercaptoenthanol, N-acetylcysteine, cysteine, riboflavin, niacin, folate, flavin adenine dinucleotide (FAD), nicotinamide adenine dinucleotide phosphate (NADP), and combination thereof. In some embodiments, a suitable redox-modulator is cysteine. In some embodiments, the cysteine is at a concentration ranging from about 0.1 mg/L to about 65 mg/L (e.g., 1-50 mg/L, 1-40 mg/L, 1-30 mg/1, 1-20 mg/L, 1-10 mg/L). In some embodiments, a suitable redox-modulator is 2-mercaptoenthanol. In some embodiments, the 2-mercaptoenthanol is at a concentration ranging from about 0.001 mM to about 0.01 mM (e.g., about 0.001-0.008 mM, about 0.001-0.007 mM, about 0.001-0.006 mM, about 0.001-0.005 mM, about 0.001-0.004 mM, about 0.001-0.003 mM, about 0.001-0.002 mM). In some embodiments, a suitable redox-modulator is N-acetylcysteine. In some embodiments, the N-acetylcysteine is at a concentration ranging from about 3 mM to about 9 mM (e.g., about 3-8 mM, about 3-7 mM, about 3-6 mM, about 3-5 mM, about 3-4 mM).

In some embodiments, a medium suitable for the present invention contains at least one growth-modulator. In some embodiments, a suitable growth-modulator is hypoxanthine. In some embodiments, the hypoxanthine is at a concentration ranging from about 0.1 mM to about 10 mM (e.g., about 0.1-9 mM, about 0.1-8 mM, about 0.1-7 mM, about 0.1-6 mM, about 0.1-5 mM, about 0.1-4 mM, about 0.1-3 mM, about 0.1-2 mM, about 0.1-1 mM). In some embodiments, a suitable growth-modulator is thymidine. In some embodiments, the thymidine is at a concentration ranging from about 1 mM to about 100 mM (e.g., about 1-90 mM, about 1-80 mM, about 1-70 mM, about 1-60 mM, about 1-50 mM, about 1-40 mM, about 1-30 mM, about 1-20 mM, about 1-10 mM).

In some embodiments, the medium has a pH ranging from about 6.8-7.5 (e.g., about 6.9-7.4, about 6.9-7.3, about 6.95-7.3, about 6.95-7.25, about 7.0-7.3, about 7.0-7.25, about 7.0-7.2, about 7.0-7.15, about 7.05-7.3, about 7.05-7.25, about 7.05-7.15, about 7.05-7.20, about 7.10-7.3, about 7.10-7.25, about 7.10-7.20, about 7.10-7.15). In some embodiments, the medium has a pH of about 6.8, 6.85, 6.9, 6.95, 7.0, 7.05, 7.1, 7.15, 7.2, 7.25, 7.3, 7.35, 7.4, 7.45, or 7.5.

In some embodiments, the culturing step of various methods described herein include a growth phase and a production phase. In some embodiments, the mammalian cells are cultured at a temperature ranging from about 30-37° C. (e.g., about 31-37° C., about 32-37° C., about 33-37° C., about 34-37° C., about 35-37° C., about 36-37° C.). In some embodiments, the mammalian cells are cultured at a temperature of approximately 30° C., 31° C., 32° C., 33° C., 34° C., 35° C., 36° C., or 37° C. Any of the temperatures described herein may be used for growth and/or production phase. In some embodiments, the mammalian cells are cultured at different temperatures during the growth phase and the production phase. In some embodiments, the mammalian cells are cultured at substantially the same temperatures during the growth phase and the production phase. Any of the medium pH described herein may be used for growth and/or production phase. In some embodiments, the medium pH for the growth phase and the production phase is different. In some embodiments, the medium pH for the growth phase and the production phase is substantially the same.

In some embodiments, the mammalian cells are maintained at a viable cell density ranging from about 1.0-50×10⁶ viable cells/mL during the production phase (e.g., about 1.0-40×10⁶ viable cells/mL, about 1.0-30×10⁶ viable cells/mL, about 1.0-20×10⁶ viable cells/mL, about 1.0-10×10⁶ viable cells/mL, about 1.0-5×10⁶ viable cells/mL, about 1.0-4.5×10⁶ viable cells/mL, about 1.0-4×10⁶ viable cells/mL, about 1.0-3.5×10⁶ viable cells/mL, about 1.0-3×10⁶ viable cells/mL, about 1.0-2.5×10⁶ viable cells/mL, about 1.0-2.0×10⁶ viable cells/mL, about 1.0-1.5×10⁶ viable cells/mL, about 1.5-10×10⁶ viable cells/mL, about 1.5-5×10⁶ viable cells/mL, about 1.5-4.5×10⁶ viable cells/mL, about 1.5-4×10⁶ viable cells/mL, about 1.5-3.5×10⁶ viable cells/mL, about 1.5-3.0×10⁶ viable cells/mL, about 1.5-2.5×10⁶ viable cells/mL, about 1.5-2.0×10⁶ viable cells/mL).

In some embodiments, the production phase is lasted for about 5-90 days (e.g., about 5-80 days, about 5-70 days, about 5-60 days, about 5-50 days, about 5-40, about 5-30 days, about 5-20 days, about 5-15 days, about 5-10 days, about 10-90 days, about 10-80 days, about 10-70 days, about 10-60 days, about 10-50 days, about 10-40 days, about 10-30 days, about 10-20 days, about 15-90 days, about 15-80 days, about 15-70 days, about 15-60 days, about 15-50 days, about 15-40 days, about 15-30 days). In some embodiments, the production phase is lasted for about 5, 10, 15, 20, 25, 30, 35, 40, 45, 50, 55, 60, 65, 70, 75, 80, 85, or 90 days.

In various embodiments, mammalian cells express a recombinant I2S protein having an amino acid sequence at least about 50% (e.g., at least about 55%, 60%, 65%, 70%, 75%, 80%, 85%, 90%, 95%, 96%, 97%, 98%, 99%) identical to SEQ ID NO:1. In some embodiments, an inventive method described herein is used to produce a recombinant I2S protein having an amino acid sequence identical to SEQ ID NO:1.

In various embodiments, mammalian cells express an FGE protein having an amino acid sequence at least about 50% (e.g., at least about 55%, 60%, 65%, 70%, 75%, 80%, 85%, 90%, 95%, 96%, 97%, 98%, 99%) identical to SEQ ID NO:5. In some embodiments, a mammalian cell expresses an FGE protein having an amino acid sequence identical to SEQ ID NO:5.

In various embodiments, mammalian cells contain one or more exogenous nucleic acids encoding the recombinant I2S protein and/or the FGE. In some embodiments, the one or more exogenous nucleic acids are integrated in the genome of the cells. In some embodiments, the one or more exogenous nucleic acids are present on one or more extra-chromosomal constructs. In some embodiments, mammalian cells used in a method of the present invention over-express the recombinant I2S protein. In some embodiments, mammalian cells used in a method of the present invention over-express the FGE.

In various embodiments, an inventive method according to the present invention further includes a step of harvesting the recombinant I2S protein.

In yet another aspect, the present invention provides a recombinant iduronate-2-sulfatase (I2S) protein produced using a method described herein. In some embodiments, the present invention provides a preparation of recombinant I2S protein, in which the recombinant I2S protein has at least about 70% (e.g., at least about 77%, 80%, 85%, 90%, 95%, 96%, 97%, 98%, 99%) conversion of the cysteine residue corresponding to Cys59 of human I2S (SEQ ID NO:1) to C_{α}-formylglycine (FGly). In some embodiments, the present invention provides a preparation of recombinant I2S protein, in which the recombinant I2S protein has substantially 100% conversion of the cysteine residue corresponding to Cys59 of human I2S (SEQ ID NO:1) to C_{α}-formylglycine (FGly). In some embodiments, the present invention provides a preparation of recombinant iduronate-2-sulfatase (I2S) protein, said recombinant I2S protein having an amino acid sequence at least about 50% (e.g., at least about 55%, 60%, 65%, 70%, 75%, 80%, 85%, 90%, 95%, 96%, 97%, 98%, 99%) identical to SEQ ID NO:1. In some embodiments, the recombinant I2S protein has an amino acid sequence identical to SEQ ID NO:1.

In some embodiments, the recombinant I2S protein has specific activity of at least about 20 U/mg, 30 U/mg, 40 U/mg, 50 U/mg, 60 U/mg, 70 U/mg, 80 U/mg, 90 U/mg, or 100 U/mg mg as determined by an in vitro sulfate release activity assay using heparin disaccharide as substrate.

Among other things, the present invention also provides a pharmaceutical composition containing a recombinant I2S protein described in various embodiments herein and a pharmaceutically acceptable carrier and a method of treating Hunter syndrome by administering into a subject in need of treatment recombinant I2S protein described herein or a pharmaceutical composition containing the same.

As used herein, the terms “I2S protein,” “I2S,” “I2S enzyme,” or grammatical equivalents, refer to a preparation of recombinant I2S protein molecules unless otherwise specifically indicated.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The Figures described below, that together make up the Drawing, are for illustration purposes only, not for limitation.

FIG. 1 depicts the amino acid sequence encoding the mature form of human iduronate-2-sulfatase (I2S) protein and indicates potential sites within the protein sequence for N-linked glycosylation and cysteine conversion.

FIG. 2 depicts exemplary construct designs for co-expression of I2S and FGE (i.e., SUMF1). (A) Expression units on separate vectors (for co-transfection or subsequent transfections); (B) Expression units on the same vector (one transfection): (1) Separate cistrons and (2) Transcriptionally linked cistrons.

FIG. 3 demonstrates exemplary expression of full length recombinant I2S by SDS-PAGE generated using cell lines grown under either serum-free or serum based cell culture conditions, as compared to an I2S reference standard.

FIG. 4 shows an exemplary peptide map for a recombinant I2S enzyme produced from the I2S-AF 2D cell line grown under serum-free culture conditions (top panel), versus a reference recombinant I2S enzyme

FIG. 5 depicts an exemplary glycan profile generated for recombinant I2S enzyme produced using the I2S-AF 2D and 4D cell lines grown under serum-free cell culture conditions as compared to a reference recombinant I2S enzyme.

FIG. 6 depicts an exemplary charge profile generated for recombinant I2S enzyme produced using the I2S-AF 2D cell line grown under serum-free cell culture conditions as compared to a reference recombinant I2S enzyme.

### DEFINITIONS

In order for the present invention to be more readily understood, certain terms are first defined. Additional definitions for the following terms and other terms are set forth throughout the specification.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc. In some embodiments, amino acids of the present invention may be provided in or used to supplement medium for cell cultures. In some embodiments, amino acids provided in or used to supplement cell culture medium may be provided as salts or in hydrate form.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Batch culture: The term “batch culture” as used herein refers to a method of culturing cells in which all the components that will ultimately be used in culturing the cells, including the medium (see definition of “medium” below) as well as the cells themselves, are provided at the beginning of the culturing process. Thus, a batch culture typically refers to a culture allowed to progress from inoculation to conclusion without refeeding the cultured cells with fresh medium. A batch culture is typically stopped at some point and the cells and/or components in the medium are harvested and optionally purified.

Bioavailability: As used herein, the term “bioavailability” generally refers to the percentage of the administered dose that reaches the blood stream of a subject.

Biologically active: As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. Biological activity can also be determined by in vitro assays (for example, in vitro enzymatic assays such as sulfate release assays). In particular embodiments, where a protein or polypeptide is biologically active, a portion of that protein or polypeptide that shares at least one biological activity of the protein or polypeptide is typically referred to as a “biologically active” portion. In some embodiments, a protein is produced and/or purified from a cell culture system, which displays biologically activity when administered to a subject. In some embodiments, a protein requires further processing in order to become biologically active. In some embodiments, a protein requires posttranslational modification such as, but is not limited to, glycosylation (e.g., sialyation), farnysylation, cleavage, folding, formylglycine conversion and combinations thereof, in order to become biologically active. In some embodiments, a protein produced as a proform (i.e. immature form), may require additional modification to become biologically active.

Bioreactor: The term “bioreactor” as used herein refers to a vessel used for the growth of a host cell culture. A bioreactor can be of any size so long as it is useful for the culturing of mammalian cells. Typically, a bioreactor will be at least 1 liter and may be 10, 100, 250, 500, 1000, 2500, 5000, 8000, 10,000, 12,0000 liters or more, or any volume in between. Internal conditions of a bioreactor, including, but not limited to pH, osmolarity, CO₂ saturation, O₂ saturation, temperature and combinations thereof, are typically controlled during the culturing period. A bioreactor can be composed of any material that suitable for holding cells in media under the culture conditions of the present invention, including glass, plastic or metal. In some embodiments, a bioreactor may be used for performing animal cell culture. In some embodiments, a bioreactor may be used for performing mammalian cell culture. In some embodiments, a bioreactor may used with cells and/or cell lines derived from such organisms as, but not limited to, mammalian cell, insect cells, bacterial cells, yeast cells and human cells. In some embodiments, a bioreactor is used for large-scale cell culture production and is typically at least 100 liters and may be 200, 500, 1000, 2500, 5000, 8000, 10,000, 12,0000 liters or more, or any volume in between. One of ordinary skill in the art will be aware of and will be able to choose suitable bioreactors for use in practicing the present invention.

Cell density: The term “cell density” as used herein refers to that number of cells present in a given volume of medium.

Cell culture or culture: These terms as used herein refer to a cell population that is gown in a medium under conditions suitable to survival and/or growth of the cell population. As will be clear to those of ordinary skill in the art, these terms as used herein may refer to the combination comprising the cell population and the medium in which the population is grown.

Cultivation: As used herein, the term “cultivation” or grammatical equvilents refers to a process of maintaining cells under conditions favoring growth or survival. The terms “cultivation” and “cell culture” or any synonyms are used inter-changeably in this application.

Culture vessel: As used herein, the term “culture vessel” refers to any container that can provide an aseptic environment for culturing cells. Exemplary culture vessels include, but are not limited to, glass, plastic, or metal containers.

Dosage form: As used herein, the terms “dosage form” and “unit dosage form” refer to a physically discrete unit of a therapeutic protein for the patient to be treated. Each unit contains a predetermined quantity of active material calculated to produce the desired therapeutic effect. It will be understood, however, that the total dosage of the composition will be decided by the attending physician within the scope of sound medical.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regiment, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses.

Enzyme replacement therapy (ERT): As used herein, the term “enzyme replacement therapy (ERT)” refers to any therapeutic strategy that corrects an enzyme deficiency by providing the missing enzyme. In some embodiments, the missing enzyme is provided by intrathecal administration. In some embodiments, the missing enzyme is provided by infusing into bloodstream. Once administered, enzyme is taken up by cells and transported to the lysosome, where the enzyme acts to eliminate material that has accumulated in the lysosomes due to the enzyme deficiency. Typically, for lysosomal enzyme replacement therapy to be effective, the therapeutic enzyme is delivered to lysosomes in the appropriate cells in target tissues where the storage defect is manifest.

Excipient: As used herein, the term “excipient” refers to any inert substance added to a drug and/or formulation for the purposes of improving its physical qualities (i.e. consistency), pharmacokinetic properties (i.e. bioavailabity), pharmacodynamic properties and combinations thereof.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fed-batch culture: The term “fed-batch culture” as used herein refers to a method of culturing cells in which additional components are provided to the culture at some time subsequent to the beginning of the culture process. The provided components typically comprise nutritional supplements for the cells which have been depleted during the culturing process. A fed-batch culture is typically stopped at some point and the cells and/or components in the medium are harvested and optionally purified.

Fragment: The term “fragment” as used herein refers to polypeptides and is defined as any discrete portion of a given polypeptide that is unique to or characteristic of that polypeptide. The term as used herein also refers to any discrete portion of a given polypeptide that retains at least a fraction of the activity of the full-length polypeptide. Preferably the fraction of activity retained is at least 10% of the activity of the full-length polypeptide. More preferably the fraction of activity retained is at least 20%, 30%, 40%, 50%, 60%, 70%, 80% or 90% of the activity of the full-length polypeptide. More preferably still the fraction of activity retained is at least 95%, 96%, 97%, 98% or 99% of the activity of the full-length polypeptide. Most preferably, the fraction of activity retained is 100% of the activity of the full-length polypeptide. The term as used herein also refers to any portion of a given polypeptide that includes at least an established sequence element found in the full-length polypeptide. Preferably, the sequence element spans at least 4-5, more preferably at least about 10, 15, 20, 25, 30, 35, 40, 45, 50 or more amino acids of the full-length polypeptide.

Gene: The term “gene” as used herein refers to any nucleotide sequence, DNA or RNA, at least some portion of which encodes a discrete final product, typically, but not limited to, a polypeptide, which functions in some aspect of a cellular process. The term is not meant to refer only to the coding sequence that encodes the polypeptide or other discrete final product, but may also encompass regions preceding and following the coding sequence that modulate the basal level of expression, as well as intervening sequences (“introns”) between individual coding segments (“exons”). In some embodiments, a gene may include regulatory sequences (e.g., promoters, enhancers, polyadenylation sequences, termination sequences, Kozak sequences, TATA box, etc.) and/or modification sequences. In some embodiments, a gene may include references to nucleic acids that do not encode proteins but rather encode functional RNA molecules such as tRNAs, RNAi-inducing agents, etc.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Genetic control element: The term “genetic control element” as used herein refers to any sequence element that modulates the expression of a gene to which it is operably linked. Genetic control elements may function by either increasing or decreasing the expression levels and may be located before, within or after the coding sequence. Genetic control elements may act at any stage of gene expression by regulating, for example, initiation, elongation or termination of transcription, mRNA splicing, mRNA editing, mRNA stability, mRNA localization within the cell, initiation, elongation or termination of translation, or any other stage of gene expression. Genetic control elements may function individually or in combination with one another.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM 120 weight residue table, a gap length penalty of 12 and a gap penalty of 4. The percent identity between two nucleotide sequences can, alternatively, be determined using the GAP program in the GCG software package using an NWSgapdna.CMP matrix. Various other sequence alignment programs are available and can be used to determine sequence identity such as, for example, Clustal.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of lysosomal storage disease as the individual being treated, who is about the same age as the individual being treated (to ensure that the stages of the disease in the treated individual and the control individual(s) are comparable).

Integrated Viable Cell Density: The term “integrated viable cell density” as used herein refers to the average density of viable cells over the course of the culture multiplied by the amount of time the culture has run. Assuming the amount of polypeptide and/or protein produced is proportional to the number of viable cells present over the course of the culture, integrated viable cell density is a useful tool for estimating the amount of polypeptide and/or protein produced over the course of the culture.

Intrathecal administration: As used herein, the term “intrathecal administration” or “intrathecal injection” refers to an injection into the spinal canal (intrathecal space surrounding the spinal cord). Various techniques may be used including, without limitation, lateral cerebroventricular injection through a burrhole or cisternal or lumbar puncture or the like. In some embodiments, “intrathecal administration” or “intrathecal delivery” according to the present invention refers to IT administration or delivery via the lumbar area or region, i.e., lumbar IT administration or delivery. As used herein, the term “lumbar region” or “lumbar area” refers to the area between the third and fourth lumbar (lower back) vertebrae and, more inclusively, the L2-S1 region of the spine.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.)

Medium: The terms as used herein refer to a solution containing nutrients which nourish growing cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. In some embodiments, medium is formulated to a pH and salt concentration optimal for cell survival and proliferation. In some embodiments, medium may be a “chemically defined medium”—a serum-free media that contains no proteins, hydrolysates or components of unknown composition. In some embodiment, chemically defined medium is free of animal-derived components and all components within the medium have a known chemical structure. In some embodiments, medium may be a “serum based medium”—a medium that has been supplemented with animal derived components such as, but not limited to, fetal calf serum, horse serum, goat serum, donkey serum and/or combinations thereof.

Metabolic waste product: The term “metabolic waste product” as used herein refers to compounds produced by the cell culture as a result of normal or non-normal metabolic processes that are in some way detrimental to the cell culture, particularly in relation to the expression or activity of a desired recombinant polypeptide or protein. For example, the metabolic waste products may be detrimental to the growth or viability of the cell culture, may decrease the amount of recombinant polypeptide or protein produced, may alter the folding, stability, glycoslyation or other post-translational modification of the expressed polypeptide or protein, or may be detrimental to the cells and/or expression or activity of the recombinant polypeptide or protein in any number of other ways. Exemplary metabolic waste products include lactate, which is produced as a result of glucose metabolism, and ammonium, which is produced as a result of glutamine metabolism. One goal of the present invention is to slow production of, reduce or even eliminate metabolic waste products in mammalian cell cultures.

Nucleic acid: As used herein, the term “nucleic acid,” in its broadest sense, refers to a compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

Osmolarity and Osmolality “Osmolality” is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of solution (1 mOsm/kg H₂O at 38° C. is equivalent to an osmotic pressure of 19 mm Hg). “Osmolarity,” by contrast, refers to the number of solute particles dissolved in 1 liter of solution. When used herein, the abbreviation “mOsm” means “milliosmoles/kg solution”.

Perfusion process: The term “perfusion process” as used herein refers to a method of culturing cells in which additional components are provided continuously or semi-continuously to the culture subsequent to the beginning of the culture process. The provided components typically comprise nutritional supplements for the cells which have been depleted during the culturing process. A portion of the cells and/or components in the medium are typically harvested on a continuous or semi-continuous basis and are optionally purified. Typically, a cell culture process involving a perfusion process is referred to as “perfusion culture.” Typically, nutritional supplements are provided in a fresh medium during a perfusion process. In some embodiments, a fresh medium may be identical or similar to the base medium used in the cell culture process. In some embodiments, a fresh medium may be different than the base medium but containing desired nutritional supplements. In some embodiments, a fresh medium is a chemically-defined medium.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. In some embodiments, a protein can sometimes include more than one polypeptide chain, for example linked by one or more disulfide bonds or associated by other means. In some embodiments, polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant protein and Recombinant polypeptide: These terms as used herein refer to a polypeptide expressed from a host cell, that has been genetically engineered to express that polypeptide. In some embodiments, a recombinant protein may be expressed in a host cell derived from an animal. In some embodiments, a recombinant protein may be expressed in a host cell derived from an insect. In some embodiments, a recombinant protein may be expressed in a host cell derived from a yeast. In some embodiments, a recombinant protein may be expressed in a host cell derived from a prokaryote. In some embodiments, a recombinant protein may be expressed in a host cell derived from an mammal. In some embodiments, a recombinant protein may be expressed in a host cell derived from a human. In some embodiments, the recombinantly expressed polypeptide may be identical or similar to a polypeptide that is normally expressed in the host cell. In some embodiments, the recombinantly expressed polypeptide may be foreign to the host cell, i.e. heterologous to peptides normally expressed in the host cell. Alternatively, in some embodiments the recombinantly expressed polypeptide can be a chimeric, in that portions of the polypeptide contain amino acid sequences that are identical or similar to polypeptides normally expressed in the host cell, while other portions are foreign to the host cell.

Replacement enzyme: As used herein, the term “replacement enzyme” refers to any enzyme that can act to replace at least in part the deficient or missing enzyme in a disease to be treated. In some embodiments, the term “replacement enzyme” refers to any enzyme that can act to replace at least in part the deficient or missing lysosomal enzyme in a lysosomal storage disease to be treated. In some embodiments, a replacement enzyme is capable of reducing accumulated materials in mammalian lysosomes or that can rescue or ameliorate one or more lysosomal storage disease symptoms. Replacement enzymes suitable for the invention include both wild-type or modified lysosomal enzymes and can be produced using recombinant and synthetic methods or purified from nature sources. A replacement enzyme can be a recombinant, synthetic, gene-activated or natural enzyme.

Seeding: The term “seeding” as used herein refers to the process of providing a cell culture to a bioreactor or another vessel for large scale cell culture production. In some embodiments a “seed culture” is used, in which the cells have been propagated in a smaller cell culture vessel, i.e. Tissue-culture flask, Tissue-culture plate, Tissue-culture roller bottle, etc., prior to seeding. Alternatively, in some embodiments, the cells may have been frozen and thawed immediately prior to providing them to the bioreactor or vessel. The term refers to any number of cells, including a single cell.

Subject: As used herein, the term “subject” means any mammal, including humans. In certain embodiments of the present invention the subject is an adult, an adolescent or an infant. Also contemplated by the present invention are the administration of the pharmaceutical compositions and/or performance of the methods of treatment in-utero.

Titer: The term “titer” as used herein refers to the total amount of recombinantly expressed polypeptide or protein produced by a cell culture divided by a given amount of medium volume.

Vector: As used herein, “vector” refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

Viable cell density: As used herein, the term “viable cell density” refers to the number of living cells per unit volume.

### DETAILED DESCRIPTION OF THE INVENTION

The present invention provides, among other things, methods and compositions for large-scale production of recombinant I2S protein using suspension culture of mammalian cells in serum-free medium. In particular, the present invention uses mammalian cells that co-express a recombinant I2S protein and a formylglycine generating enzyme (FGE).

Various aspects of the invention are described in further detail in the following subsections. The use of subsections is not meant to limit the invention. Each subsection may apply to any aspect of the invention. In this application, the use of “or” means “and/or” unless stated otherwise.

Iduronate-2-sulfatase (I2S)

As used herein, an I2S protein is any protein or a portion of a protein that can substitute for at least partial activity of naturally-occurring Iduronate-2-sulfatase (I2S) protein or rescue one or more phenotypes or symptoms associated with I2S-deficiency. As used herein, the terms “an I2S enzyme” and “an I2S protein”, and grammatical equivalents, are used inter-changeably.

Typically, the human I2S protein is produced as a precursor form. The precursor form of human I2S contains a signal peptide (amino acid residues 1-25 of the full length precursor), a pro-peptide (amino acid residues 26-33 of the full length precursor), and a chain (residues 34-550 of the full length precursor) that may be further processed into the 42 kDa chain (residues 34-455 of the full length precursor) and the 14 kDa chain (residues 446-550 of the full length precursor). Typically, the precursor form is also referred to as full-length precursor or full-length I2S protein, which contains 550 amino acids. The amino acid sequences of the mature form (SEQ ID NO:1) having the signal peptide removed and full-length precursor (SEQ ID NO:2) of a typical wild-type or naturally-occurring human I2S protein are shown in Table 1. The signal peptide is underlined. In addition, the amino acid sequences of human I2S protein isoform a and b precursor are also provided in Table 1, SEQ ID NO:3 and 4, respectively.

TABLE 1
Human Iduronate-2-sulfatase

| | |
|---|---|
| Mature Form | SETQANSTTDALNVLLIIVDDLRPSLGCYGDKLVRSPNIDQLASHSLLFQNAFA |
| | QQAVCAPSRVSFLTGRRPDTTRLYDFNSYWRVHAGNFSTIPQYFKENGYVTMSV |
| | GKVFHPGISSNHTDDSPYSWSFPPYHPSSEKYENTKTCRGPDGELHANLLCPVD |
| | VLDVPEGTLPDKQSTEQAIQLLEKMKTSASPFFLAVGYHKPHIPFRYPKEFQKL |
| | YPLENITLAPDPEVPDGLPPVAYNPWMDIRQREDVQALNISVPYGPIPVDFQRK |
| | IRQSYFASVSYLDTQVGRLLSALDDLQLANSTIIAFTSDHGWALGEHGEWAKYS |
| | NFDVATHVPLIFYVPGRTASLPEAGEKLFPYLDPFDSASQLMEPGRQSMDLVEL |
| | VSLFPTLAGLAGLQVPPRCPVPSFHVELCREGKNLLKHFRFRDLEEDPYLPGNP |
| | RELIAYSQYPRPSDIPQWNSDKPSLKDIKIMGYSIRTIDYRYTVWVGFNPDEFL |
| | ANFSDIHAGELYFVDSDPLQDHNMYNDSQGGDLFQLLMP |
| | (SEQ ID NO: 1) |
| Full-Length | MPPPRTGRGLLWLGLVLSSVCVALGSETQANSTTDALNVLLIIVDDLRPSLGCY |
| Precursor | GDKLVRSPNIDQLASHSLLFQNAFAQQAVCAPSRVSFLTGRRPDTTRLYDFNSY |
| (Isoform a) | WRVHAGNFSTIPQYFKENGYVTMSVGKVFHPGISSNHTDDSPYSWSFPPYHPSS |
| | EKYENTKTCRGPDGELHANLLCPVDVLDVPEGTLPDKQSTEQAIQLLEKMKTSA |
| | SPFFLAVGYHKPHIPFRYPKEFQKLYPLENITLAPDPEVPDGLPPVAYNPWMDI |
| | RQREDVQALNISVPYGPIPVDFQRKIRQSYFASVSYLDTQVGRLLSALDDLQLA |
| | NSTITAFTSDHGWALGEHGEWAKYSNFDVATHVPLIFYVPGRTASLPEAGEKLF |
| | PYLDPFDSASQLMEPGRQSMDLVELVSLFPTLAGLAGLQVPPRCPVPSFHVELC |
| | REGKNLLKHFRFRDLEEDPYLPGNPRELIAYSQYPRPSDIPQWNSDKPSLKDIK |
| | IMGYSIRTIDYRYTVWVGFNPDEFLANFSDIHAGELYFVDSDPLQDHNMYNDSQ |
| | GGDLFQLLMP |
| | (SEQ ID NO: 2) |
| Isoform b Precursor | MPPPRTGRGLLWLGLVLSSVCVALGSETQANSTTDALNVLLIIVDDLRPSLGCY |
| | GDKLVRSPNIDQLASHSLLFQNAFAQQAVCAPSRVSFLTGRRPDTTRLYDFNSY |
| | WRVHAGNFSTIPQYFKENGYVTMSVGKVFHPGISSNHTDDSPYSWSFPPYHPSS |
| | EKYENTKTCRGPDGELHANLLCPVDVLDVPEGTLPDKQSTEQAIQLLEKMKTSA |
| | SPFFLAVGYHKPHIPFRYPKEFQKLYPLENITLAPDPEVPDGLPPVAYNPWMDI |
| | RQREDVQALNISVPYGPIPVDFQEDQSSTGFRLKTSSTRKYK |
| | (SEQ ID NO: 3) |
| Isoform c Precursor | MPPPRTGRGLLWLGLVLSSVCVALGSETQANSTTDALNVLLIIVDDLRPSLGCY |
| | GDKLVRSPNIDQLASHSLLFQNAFAQQAVCAPSRVSFLTGRRPDTTRLYDFNSY |
| | WRVHAGNFSTIPQYFKENGYVTMSVGKVFHPGISSNHTDDSPYSWSFPPYHPSS |
| | EKYENTKTCRGPDGELHANLLCPVDVLDVPEGTLPDKQSTEQAIQLLEKMKTSA |
| | SPFFLAVGYHKPHIPFRYPKEFQKLYPLENITLAPDPEVPDGLPPVAYNPWMDI |
| | RQREDVQALNISVPYGPIPVDFQRKIRQSYFASVSYLDTQVGRLLSALDDLQLA |
| | NSTIIAFTSDHGFLMRTNT |
| | (SEQ ID No: 4) |

Thus, in some embodiments, an I2S enzyme is mature human I2S protein (SEQ ID NO:1). As disclosed herein, SEQ ID NO:1 represents the canonical amino acid sequence for the human I2S protein. In some embodiments, the I2S protein may be a splice isoform and/or variant of SEQ ID NO:1, resulting from transcription at an alternative start site within the 5′ UTR of the I2S gene. In some embodiments, a suitable replacement enzyme may be a homologue or an analogue of mature human I2S protein. For example, a homologue or an analogue of mature human I2S protein may be a modified mature human I2S protein containing one or more amino acid substitutions, deletions, and/or insertions as compared to a wild-type or naturally-occurring I2S protein (e.g., SEQ ID NO:1), while retaining substantial I2S protein activity. Thus, in some embodiments, a replacement enzyme suitable for the present invention is substantially homologous to mature human I2S protein (SEQ ID NO:1). In some embodiments, a replacement enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1. In some embodiments, a replacement enzyme suitable for the present invention is substantially identical to mature human I2S protein (SEQ ID NO:1). In some embodiments, a replacement enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:1. In some embodiments, a replacement enzyme suitable for the present invention contains a fragment or a portion of mature human I2S protein.

Alternatively, an I2S enzyme is full-length I2S protein. In some embodiments, an I2S enzyme may be a homologue or an analogue of full-length human I2S protein. For example, a homologue or an analogue of full-length human I2S protein may be a modified full-length human I2S protein containing one or more amino acid substitutions, deletions, and/or insertions as compared to a wild-type or naturally-occurring full-length I2S protein (e.g., SEQ ID NO:2), while retaining substantial I2S protein activity. Thus, in some embodiments, an I2S enzyme is substantially homologous to full-length human I2S protein (SEQ ID NO:2). In some embodiments, an I2S enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:2. In some embodiments, an I2S enzyme suitable for the present invention is substantially identical to SEQ ID NO:2. In some embodiments, an I2S enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:2. In some embodiments, an I2S enzyme suitable for the present invention contains a fragment or a portion of full-length human I2S protein. As used herein, a full-length I2S protein typically contains signal peptide sequence.

In some embodiments, an I2S enzyme suitable for the present invention is human I2S isoform a protein. In some embodiments, a suitable I2S enzyme may be a homologue or an analogue of human I2S isoform a protein. For example, a homologue or an analogue of human I2S isoform a protein may be a modified human I2S isoform a protein containing one or more amino acid substitutions, deletions, and/or insertions as compared to a wild-type or naturally-occurring human I2S isoform a protein (e.g., SEQ ID NO:3), while retaining substantial I2S protein activity. Thus, in some embodiments, an I2S enzyme is substantially homologous to human I2S isoform a protein (SEQ ID NO:3). In some embodiments, an I2S enzyme has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:3. In some embodiments, an I2S enzyme is substantially identical to SEQ ID NO:3. In some embodiments, an I2S enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:3. In some embodiments, an I2S enzyme suitable for the present invention contains a fragment or a portion of human I2S isoform a protein. As used herein, a human I2S isoform a protein typically contains a signal peptide sequence.

In some embodiments, an I2S enzyme is human I2S isoform b protein. In some embodiments, an I2S enzyme may be a homologue or an analogue of human I2S isoform b protein. For example, a homologue or an analogue of human I2S isoform b protein may be a modified human I2S isoform b protein containing one or more amino acid substitutions, deletions, and/or insertions as compared to a wild-type or naturally-occurring human I2S isoform b protein (e.g., SEQ ID NO:4), while retaining substantial I2S protein activity. Thus, In some embodiments, an I2S enzyme is substantially homologous to human I2S isoform b protein (SEQ ID NO:4). In some embodiments, an I2S enzyme has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:4. In some embodiments, an I2S enzyme is substantially identical to SEQ ID NO:4. In some embodiments, an I2S enzyme has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:4. In some embodiments, an I2S enzyme suitable for the present invention contains a fragment or a portion of human I2S isoform b protein. As used herein, a human I2S isoform b protein typically contains a signal peptide sequence.

Homologues or analogues of human I2S proteins can be prepared according to methods for altering polypeptide sequence known to one of ordinary skill in the art such as are found in references that compile such methods. In some embodiments, conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In some embodiments, a “conservative amino acid substitution” refers to an amino acid substitution that does not alter the relative charge or size characteristics of the protein in which the amino acid substitution is made.

In some embodiments, I2S enzymes contain a moiety that binds to a receptor on the surface of cells to facilitate cellular uptake and/or lysosomal targeting. For example, such a receptor may be the cation-independent mannose-6-phosphate receptor (CI-MPR) which binds the mannose-6-phosphate (M6P) residues. In addition, the CI-MPR also binds other proteins including IGF-II. A suitable lysosomal targeting moiety can be IGF-I, IGF-II, RAP, p97, and variants, homologues or fragments thereof (e.g., including those peptide having a sequence at least 70%, 75%, 80%, 85%, 90%, or 95% identical to a wild-type mature human IGF-I, IGF-II, RAP, p97 peptide sequence). In some embodiments, a suitable receptor that the M6P residues bind may be cation-dependent.

### Formylglycine Generating Enzyme (FGE)

Typically, the enzyme activity of I2S is influenced by a post-translational modification of a conserved cysteine (e.g., corresponding to amino acid 59 of the mature human I2S (SEQ ID NO:1)) to formylglycine, which is also referred to as 2-amino-3-oxopropionic acid, or oxo-alanine. This post-translational modification generally occurs in the endoplasmic reticulum during protein synthesis and is catalyzed by Formylglycine Generating Enzyme (FGE). The specific enzyme activity of I2S is typically positively correlated with the extent to which the I2S has the formylglycine modification. For example, an I2S protein preparation that has a relatively high amount of formylglycine modification typically has a relatively high specific enzyme activity; whereas an I2S protein preparation that has a relatively low amount of formylglycine modification typically has a relatively low specific enzyme activity.

Thus, cells suitable for producing recombinant I2S protein according to the present invention typically express FGE protein. In some embodiments, suitable cells express an endogenous FGE protein. In some embodiments, suitable cells are engineered to express an exogenous or recombinant Formylglycine Generating Enzyme (FGE) in combination with recombinant I2S. In some embodiments, suitable cells are engineered to activate an endogenous FGE gene such that the expression level or activity of the FGE protein is increased.

Typically, the human FGE protein is produced as a precursor form. The precursor form of human FGE contains a signal peptide (amino acid residues 1-33 of the full length precursor) and a chain (residues 34-374 of the full length precursor). Typically, the precursor form is also referred to as full-length precursor or full-length FGE protein, which contains 374 amino acids. The amino acid sequences of the mature form (SEQ ID NO:5) having the signal peptide removed and full-length precursor (SEQ ID NO:6) of a typical wild-type or naturally-occurring human FGE protein are shown in Table 2.

TABLE 2
Human Formylglycine Generating Enzyme (FGE)

| | |
|---|---|
| Mature Form | SQEAGTGAGAGSLAGSCGCGTPQRPGAHGSSAAAHRYSREANAPGPVPGERQLA |
| | HSKMVPIPAGVFTMGTDDPQIKQDGEAPARRVTIDAFYMDAYEVSNTEFEKFVN |
| | STGYLTEAEKFGDSFVFEGMLSEQVKTNIQQAVAAAPWWLPVKGANWRHPEGPD |
| | STILHRPDHPVLHVSWNDAVAYCTWAGKRLPTEAEWEYSCRGGLHNRLFPWGNK |
| | LQPKGQHYANIWQGEFPVTNTGEDGFQGTAPVDAFPPNGYGLYNIVGNAWEWTS |
| | DWWTVHHSVEETLNPKGPPSGKDRVKKGGSYMCHRSYCYRYRCAARSQNTPDSS |
| | ASNLGFRCAADRLPTMD |
| | (SEQ ID NO: 5) |
| Full-Length | MAAPALGLVCGRCPELGLVLLLLLLSLLCGAAGSQEAGTGAGAGSLAGSCGCGT |
| Precursor | PQRPGAHGSSAAAHRYSREANAPGPVPGERQLAHSKMVPIPAGVFTMGTDDPQI |
| | KQDGEAPARRVTIDAFYMDAYEVSNTEFEKFVNSTGYLTEAEKFGDSFVFEGML |
| | SEQVKTNIQQAVAAAPWWLPVKGANWRHPEGPDSTILHRPDHPVLHVSWNDAVA |
| | YCTWAGKRLPTEAEWEYSCRGGLHNRLFPWGNKLQPKGQHYANIWQGEFPVTNT |
| | GEDGFQGTAPVDAFPPNGYGLYNIVGNAWEWTSDWWTVHHSVEETLNPKGPPSG |
| | KDRVKKGGSYMCHRSYCYRYRCAARSQNTPDSSASNLGFRCAADRLPTMD |
| | (SEQ ID NO: 6) |

Thus, in some embodiments, an FGE enzyme suitable for the present invention is mature human FGE protein (SEQ ID NO:5). In some embodiments, a suitable FGE enzyme may be a homologue or an analogue of mature human FGE protein. For example, a homologue or an analogue of mature human FGE protein may be a modified mature human FGE protein containing one or more amino acid substitutions, deletions, and/or insertions as compared to a wild-type or naturally-occurring FGE protein (e.g., SEQ ID NO:5), while retaining substantial FGE protein activity. Thus, in some embodiments, an FGE enzyme suitable for the present invention is substantially homologous to mature human FGE protein (SEQ ID NO:5). In some embodiments, an FGE enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:5. In some embodiments, an FGE enzyme suitable for the present invention is substantially identical to mature human FGE protein (SEQ ID NO:5). In some embodiments, an FGE enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:5. In some embodiments, an FGE enzyme suitable for the present invention contains a fragment or a portion of mature human FGE protein.

Alternatively, an FGE enzyme suitable for the present invention is full-length FGE protein. In some embodiments, an FGE enzyme may be a homologue or an analogue of full-length human FGE protein. For example, a homologue or an analogue of full-length human FGE protein may be a modified full-length human FGE protein containing one or more amino acid substitutions, deletions, and/or insertions as compared to a wild-type or naturally-occurring full-length FGE protein (e.g., SEQ ID NO:6), while retaining substantial FGE protein activity. Thus, in some embodiments, an FGE enzyme suitable for the present invention is substantially homologous to full-length human FGE protein (SEQ ID NO:6). In some embodiments, an FGE enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:4. In some embodiments, an FGE enzyme suitable for the present invention is substantially identical to SEQ ID NO:6. In some embodiments, an FGE enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:6. In some embodiments, an FGE enzyme suitable for the present invention contains a fragment or a portion of full-length human FGE protein. As used herein, a full-length FGE protein typically contains signal peptide sequence.

Exemplary nucleic acid sequences and amino acid sequences encoding exemplary FGE proteins are disclosed US Publication No. 20040229250, the entire contents of which is incorporated herein by reference.

### Host Cells

As used herein, the term “host cells” refers to cells that can be used to produce recombinant I2S enzyme. In particular, host cells are suitable for producing recombinant I2S enzyme at a large scale. In some embodiments, host cells are able to produce I2S enzyme in an amount of or greater than about 5 picogram/cell/day (e.g., greater than about 10, 15, 20, 25, 30, 35, 40, 45, 50, 55, 60, 65, 70, 75, 80, 85, 90, 95, or 100 picogram/cell/day). In some embodiments, host cells are able to produce I2S enzyme in an amount ranging from about 5-100 picogram/cell/day (e.g., about 5-90 picogram/cell/day, about 5-80 picogram/cell/day, about 5-70 picogram/cell/day, about 5-60 picogram/cell/day, about 5-50 picogram/cell/day, about 5-40 picogram/cell/day, about 5-30 picogram/cell/day, about 10-90 picogram/cell/day, about 10-80 picogram/cell/day, about 10-70 picogram/cell/day, about 10-60 picogram/cell/day, about 10-50 picogram/cell/day, about 10-40 picogram/cell/day, about 10-30 picogram/cell/day, about 20-90 picogram/cell/day, about 20-80 picogram/cell/day, about 20-70 picogram/cell/day, about 20-60 picogram/cell/day, about 20-50 picogram/cell/day, about 20-40 picogram/cell/day, about 20-30 picogram/cell/day). In some embodiments, a suitable host cell is not a endosomal acidification-deficient cell.

Suitable host cells can be derived from a variety of organisms, including, but not limited to, mammals, plants, birds (e.g., avian systems), insects, yeast, and bacteria. In some embodiments, host cells are mammalian cells. Any mammalian cell susceptible to cell culture, and to expression of polypeptides, may be utilized in accordance with the present invention as a host cell. Non-limiting examples of mammalian cells that may be used in accordance with the present invention include human embryonic kidney 293 cells (HEK293), HeLa cells; BALB/c mouse myeloma line (NSO/l, ECACC No: 85110503); human retinoblasts (PER.C6 (CruCell, Leiden, The Netherlands)); monkey kidney CV1 line transformed by SV40 (COS-7, ATCC CRL 1651); human fibrosarcomacell line (e.g., HT-1080); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells +/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; a human hepatoma line (Hep G2), human cell line CAP and AGEl.HN, and Glycotope's panel.

Additionally, any number of available hybridoma cell lines may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

In some embodiments, host cells are non-mammalian cells. Non-limiting examples of non-mammalian host cells suitable for the present invention include cells and cell lines derived from Pichia pastoris, Pichia methanolica, Pichia angusta, Schizosacccharomyces pombe, Saccharomyces cerevisiae, and Yarrowia lipolytica for yeast; Sodoptera frugiperda, Trichoplusis ni, Drosophila melangoster and Manduca sexta for insects; and Escherichia coli, Salmonella typhimurium, Bacillus subtilis, Bacillus lichenifonnis, Bacteroides fragilis, Clostridia perfringens, Clostridia difficile for bacteria; and Xenopus Laevis from amphibian.

### Vectors and Nucleic Acid Constructs

Various nucleic acid constructs can be used to express I2S and/or FGE enzyme described herein in host cells. A suitable vector construct typically includes, in addition to I2S and/or FGE protein-encoding sequences (also referred to as I2S or FGE transgene), regulatory sequences, gene control sequences, promoters, non-coding sequences and/or other appropriate sequences for expression of the protein and, optionally, for replication of the construct. Typically, the coding region is operably linked with one or more of these nucleic acid components.

“Regulatory sequences” typically refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, 5′ untranslated sequences, translation leader sequences, introns, and 3′ untranslated sequences such as polyadenylation recognition sequences. Sometimes, “regulatory sequences” are also referred to as “gene control sequences.”

“Promoter” typically refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions.

The “3′ non-coding sequences” typically refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The “translation leader sequence” or “5′ non-coding sequences” typically refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency.

Typically, the term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (i.e., that the coding sequence is under the transcriptional control of the promoter). Coding sequences can be operatively linked to regulatory sequences in sense or antisense orientation.

The coding region of a transgene may include one or more silent mutations to optimize codon usage for a particular cell type. For example, the codons of an I2S transgene may be optimized for expression in a vertebrate cell. In some embodiments, the codons of an I2S transgene may be optimized for expression in a mammalian cell. In some embodiments, the codons of an I2S transgene may be optimized for expression in a human cell.

Optionally, a construct may contain additional components such as one or more of the following: a splice site, an enhancer sequence, a selectable marker gene under the control of an appropriate promoter, an amplifiable marker gene under the control of an appropriate promoter, and a matrix attachment region (MAR) or other element known in the art that enhances expression of the region where it is inserted.

Once transfected or transduced into host cells, a suitable vector can express extrachromosomally (episomally) or integrate into the host cell's genome.

In some embodiments, a DNA construct that integrates into the cell's genome, it need include only the transgene nucleic acid sequences. In that case, the express of the transgene is typically controlled by the regulatory sequences at the integration site. Optionally, it can include additional various regulatory sequences described herein.

### Culture Medium

The term “medium” and “culture medium” as used herein refers to a general class of solution containing nutrients suitable for maintaining and/or growing cells in vitro. Typically, medium solutions provide, without limitation, essential and nonessential amino acids, vitamins, energy sources, lipids, and trace elements required by the cell for at least minimal growth and/or survival. In other embodiments, the medium may contain an amino acid(s) derived from any source or method known in the art, including, but not limited to, an amino acid(s) derived either from single amino acid addition(s) or from a peptone or protein hydrolysate addition(s) (including animal or plant source(s)). Vitamins such as, but not limited to, Biotin, Pantothenate, Choline Chloride, Folic Acid, Myo-Inositol, Niacinamide, Pyridoxine, Riboflavin, Vitamin B12, Thiamine, Putrescine and/or combinations thereof. Salts such as, but not limited to, CaCl₂, KCl, MgCl₂, NaCl, Sodium Phosphate Monobasic, Sodium Phosphate Dibasic, Sodium Selenite, CuSO₄, ZnCl₂ and/or combinations thereof. Fatty acids such as, but not limited to, Arachidonic Acid, Linoleic Acid, Oleic Acid, Lauric Acid, Myristic Acid, as well as Methyl-beta-Cyclodextrin and/or combinations thereof). In some embodiments, medium comprises additional components such as glucose, glutamine, Na-pyruvate, insulin or ethanolamine, a protective agent such as Pluronic F68. In some embodiments, the medium may also contain components that enhance growth and/or survival above the minimal rate, including hormones and growth factors. Medium may also comprise one or more buffering agents. The buffering agents may be designed and/or selected to maintain the culture at a particular pH (e.g., a physiological pH, (e.g., pH 6.8 to pH 7.4)). A variety of buffers suitable for culturing cells are known in the art and may be used in the methods. Suitable buffers (e.g., bicarbonate buffers, HEPES buffer, Good's buffers, etc.) are those that have the capacity and efficiency for maintaining physiological pH despite changes in carbon dioxide concentration associated with cellular respiration. The solution is preferably formulated to a pH and salt concentration optimal for cell survival and proliferation.

In some embodiments, medium may be a chemically defined medium. As used herein, the term “chemically-defined nutrient medium” refers to a medium of which substantially all of the chemical components are known. In some embodiments, a chemically defined nutrient medium is free of animal-derived components. In some cases, a chemically-defined medium comprises one or more proteins (e.g., protein growth factors or cytokines.) In some cases, a chemically-defined nutrient medium comprises one or more protein hydrolysates. In other cases, a chemically-defined nutrient medium is a protein-free media, i.e., a serum-free media that contains no proteins, hydrolysates or components of unknown composition.

Typically, a chemically defined medium can be prepared by combining various individual components such as, for example, essential and nonessential amino acids, vitamins, energy sources, lipids, salts, buffering agents, and trace elements, at predetermined weight or molar percentages or ratios. Exemplary serum-free, in particular, chemically-defined media are described in US Pub. No. 2006/0148074, the disclosure of which is hereby incorporated by reference.

In some embodiments, a chemically defined medium suitable for the present invention is a commercially available medium such as, but not limited to, Dulbecco's Modified Eagle's Medium (DMEM), DMEM F12 (1:1), Ham's Nutrient mixture F-10, Roswell Park Memorial Institute Medium (RPMI), MCDB 131, William's Medium E, CD CHO medium (Invitrogen), CD 293 medium (Invitrogen), EX-Cell CDCHO, Ex-Cell CDCHO Fusion, CD-OptiCHO, CD-FortiCHO, CDM4CHO, CD1000, BalanCD-CHO, IS-CHO-CD, CD Hybridoma, CD-DG44. In some embodiments, a chemically defined medium suitable for the present invention is a mixture of one or more commercially available chemically defined mediums. In various embodiments, a suitable medium is a mixture of two, three, four, five, six, seven, eight, nine, ten, or more commercially available chemically defined media. In some embodiments, each individual commercially available chemically defined medium (e.g., such as those described herein) constitutes, by weight, 1%, 2.5%, 5%, 7.5%, 10%, 12.5%, 15%, 20%, 25%, 30%, 35%, 40%, 45%, 50%, 55%, 60%, 65%, 70%, 75%, 80%, 85%, 90%, 95%, or more, of the mixture. Ratios between each individual component medium may be determined by relative weight percentage present in the mixture.

In some embodiments, a chemically defined medium may be supplemented by one or more animal derived components. Such animal derived components include, but are not limited to, fetal calf serum, horse serum, goat serum, donkey serum, human serum, and serum derived proteins such as albumins (e.g., bovine serum albumin or human serum albumin).

Redox-Modulators

In some embodiments, a suitable medium contains one or more redox-modulators. Without wishing to be bound by particular theory, it is contemplated that redox-modulators may improve the production and/or activity of I2S, leading to recombinant I2S compositions having high levels of active enzyme. As used herein, a “redox-modulator” is a molecule (e.g., small-molecule, polypeptide, etc.) that influences the likelihood that a constituent in a mixture will acquire electrons and thereby be reduced. A redox-modulator may increase or decrease the likelihood that a constituent in the mixture will acquire electrons and thereby be reduced. In some embodiments, a redox-modulator may already be present in a medium, e.g., when a chemically-defined medium is obtained from a commercially available source, or may be provided as an additive to the medium. In some cases, a medium according to the invention contains two or more redox-modulators. Non-limiting examples of redox-modulators include glutathione, glucose-6-phosphate, carnosine, carnosol, sulforaphane, tocopherol, ascorbate, dehydroascorbate, selenium, 2-mercaptoenthanol, N-acetylcysteine, cysteine, riboflavin, niacin, folate, flavin adenine dinucleotide (FAD), dithiothreitol and nicotinamide adenine dinucleotide phosphate (NADP). Other appropriate redox-modulators will be apparent to the skilled artisan.

In some embodiments, cysteine is added to, or present in, a medium of the invention. Cysteine may be present at various concentrations. In some embodiments, the concentration of cysteine in the medium is in a range of about 0.1 mg/L to about 10 mg/L, about 1 mg/L to about 25 mg/L, about 10 mg/L to about 50 mg/L, about 25 mg/L to about 65 mg/L, about 10 mg/L to about 100 mg/L, or about 25 mg/L to about 250 mg/L. In some embodiments, the cysteine is at a concentration ranging from about 0.1 mg/L to about 65 mg/L (e.g., 1-50 mg/L, 1-40 mg/L, 1-30 mg/1, 1-20 mg/L, 1-10 mg/L). In some cases, the concentration of cysteine in the medium is up to about 0.1 mg/L, about 1 mg/L, about 5 mg/L, about 10 mg/L, about 20 mg/L, about 25 mg/L, about 50 mg/L, about 65 mg/L, about 75 mg/L, about 100 mg/L, or more.

In some embodiments, 2-mercaptoenthanol is added to, or present in, a medium of the invention. Various concentrations may be used. In some embodiments, the concentration of 2-mercaptoenthanol is in a range of about 0.1 nM to about 0.001 mM, about 0.001 mM to about 0.01 mM, about 0.001 mM to about 0.1 mM, about 0.01 mM to about 0.1 mM, about 0.01 mM to about 1 mM. In some cases, the concentration of 2-mercaptoenthanol in up to about 0.1 nM, about 0.001 mM, about 0.01 mM, about 0.1 mM, about 1 mM or more. In some embodiments, the 2-mercaptoenthanol is at a concentration ranging from about 0.001 mM to about 0.01 mM (e.g., about 0.001-0.008 mM, about 0.001-0.007 mM, about 0.001-0.006 mM, about 0.001-0.005 mM, about 0.001-0.004 mM, about 0.001-0.003 mM, about 0.001-0.002 mM).

In some embodiments, N-acetylcysteine is added to, or present in, a medium of the invention. Various concentrations may be used. In some embodiments, the concentration of the N-acetylcysteine may be in a range of about 0.1 mM to about 1 mM, about 1 mM to about 10 mM, about 3 mM to about 9 mM, about 1 mM to about 50 mM, or about 10 mM to about 50 mM. In some embodiments, the N-acetylcysteine is at a concentration ranging from about 3 mM to about 9 mM (e.g., about 3-8 mM, about 3-7 mM, about 3-6 mM, about 3-5 mM, about 3-4 mM). In some embodiments, the concentration of the N-acetylcysteine may up to about 0.1 mM, about 1 mM, about 3 mM, about 9 mM, about 10 mM, about 20 mM, about 30 mM, about 40 mM, about 50 mM, or more.

Growth-Modulators

In some embodiments, a medium may contain one or more growth-modulators to improve the production of I2S. As used herein, the term “growth-modulator” refers to a molecule that affects the growth of a cell. A growth-modulator can increase cell growth by, e.g., enhancing or inducing cell proliferation, cell cycle progression, or decrease cell growth by, e.g., promoting cell cycle arrest. While commercially available mediums often comprise a multitude of different growth-modulators, in some cases it is desirable to provide additional growth modulators to the nutrient medium. Therefore, in some embodiments, one or more growth-modulators are added to the medium.

In some cases, a growth-modulator suitable for the invention includes hypoxanthine. In some embodiments, hypoxanthine is at a concentration in a range of about 0.01 mM to about 0.1 mM, about 0.1 mM to about 1 mM, about 0.1 mM to about 10 mM, about 1 mM to about 10 mM, about 0.1 mM to about 100 mM. In some embodiments, the hypoxanthine is at a concentration ranging from about 0.1 mM to about 10 mM (e.g., about 0.1-9 mM, about 0.1-8 mM, about 0.1-7 mM, about 0.1-6 mM, about 0.1-5 mM, about 0.1-4 mM, about 0.1-3 mM, about 0.1-2 mM, about 0.1-1 mM). In some cases, hypoxanthine is at a concentration of about 0.01 mM, about 0.1 mM, about 1 mM, about 10 mM, about 20 mM, about 30 mM, about 40 mM, about 50 mM, about 60 mM, about 70 mM, about 80 mM, about 90 mM, about 100 mM or more.

In some cases, a growth-modulator suitable for the invention includes thymidine. In some embodiments, the thymidine is at a concentration in a range of about 0.01 mM to about 0.1 mM, about 0.1 mM to about 1 mM, about 0.1 mM to about 10 mM, about 1 mM to about 10 mM, about 0.1 mM to about 100 mM, about 1 mM to about 100 mM. In some embodiments, the thymidine is at a concentration ranging from about 1 mM to about 100 mM (e.g., about 1-90 mM, about 1-80 mM, about 1-70 mM, about 1-60 mM, about 1-50 mM, about 1-40 mM, about 1-30 mM, about 1-20 mM, about 1-10 mM). In some embodiments, thymidine is at a concentration of about 0.01 mM, about 0.1 mM, about 1 mM, about 10 mM, about 20 mM, about 30 mM, about 40 mM, about 50 mM, about 60 mM, about 70 mM, about 80 mM, about 90 mM, about 100 mM or more.

### Culture Conditions

The present invention provides a method of producing recombinant I2S at a large scale. Typical large-scale procedures for producing a recombinant polypeptide of interest include batch cultures and fed-batch cultures. Batch culture processes traditionally comprise inoculating a large-scale production culture with a seed culture of a particular cell density, growing the cells under conditions (e.g., suitable culture medium, pH, and temperature) conducive to cell growth, viability, and/or productivity, harvesting the culture when the cells reach a specified cell density, and purifying the expressed polypeptide. Fed-batch culture procedures include an additional step or steps of supplementing the batch culture with nutrients and other components that are consumed during the growth of the cells. In some embodiments, a large-scale production method according to the present invention uses a fed-batch culture system.

Culture Initiation

Typically, a desired cell expressing I2S protein is first propagated in an initial culture by any of the variety of methods well-known to one of ordinary skill in the art. The cell is typically propagated by growing it at a temperature and in a medium that is conducive to the survival, growth and viability of the cell. The initial culture volume can be of any size, but is often smaller than the culture volume of the production bioreactor used in the final production, and frequently cells are passaged several times of increasing culture volume prior to seeding the production bioreactor. The cell culture can be agitated or shaken to increase oxygenation of the medium and dispersion of nutrients to the cells. Alternatively or additionally, special sparging devices that are well known in the art can be used to increase and control oxygenation of the culture.

The starting cell density can be chosen by one of ordinary skill in the art. In accordance with the present invention, the starting cell density can be as low as a single cell per culture volume. In some embodiments, starting cell densities can range from about 1×10² viable cells per mL to about 1×10³, 1×10⁴, 1×10⁵ viable cells per mL and higher.

Initial and intermediate cell cultures may be grown to any desired density before seeding the next intermediate or final production bioreactor. In some embodiments, final viability before seeding the production bioreactor is greater than about 70%, 75%, 80%, 85%, 90%, 95%, or more. The cells may be removed from the supernatant, for example, by low-speed centrifugation. It may also be desirable to wash the removed cells with a medium before seeding the next bioreactor to remove any unwanted metabolic waste products or medium components. The medium may be the medium in which the cells were previously grown or it may be a different medium or a washing solution selected by the practitioner of the present invention.

The cells may then be diluted to an appropriate density for seeding the production bioreactor. In some embodiments, the cells are diluted into the same medium that will be used in the production bioreactor. Alternatively, the cells can be diluted into another medium or solution, depending on the needs and desires of the practitioner of the present invention or to accommodate particular requirements of the cells themselves, for example, if they are to be stored for a short period of time prior to seeding the production bioreactor.

Growth Phase

Typically, once the production bioreactor has been seeded as described above, the cell culture is maintained in the initial growth phase under conditions conducive to the survival, growth and viability of the cell culture. In accordance with the present invention, the production bioreactor can be any volume that is appropriate for large-scale production of proteins. See the “Bioreactor” subsection below.

The temperature of the cell culture in the growth phase is selected based primarily on the range of temperatures at which the cell culture remains viable. The temperature of the growth phase may be maintained at a single, constant temperature, or within a range of temperatures. For example, the temperature may be steadily increased or decreased during the growth phase. In general, most mammalian cells grow well within a range of about 25° C. to 42° C. (e.g., 30° C. to 40° C., about 30° C. to 37° C., about 35° C. to 40° C.). In some embodiments, the mammalian cells are cultured at a temperature ranging from about 30-37° C. (e.g., about 31-37° C., about 32-37° C., about 33-37° C., about 34-37° C., about 35-37° C., about 36-37° C.). Typically, during the growth phase, cells grow at about 28° C., about 30° C., about 31° C., about 32° C., about 33° C., about 34° C., about 35° C., about 36° C., about 37° C., about 38° C., about 39° C., about 40° C.

The cells may be grown during the initial growth phase for a greater or lesser amount of time, depending on the needs of the practitioner and the requirement of the cells themselves. In one embodiment, the cells are grown for a period of time sufficient to achieve a viable cell density that is a given percentage of the maximal viable cell density that the cells would eventually reach if allowed to grow undisturbed. For example, the cells may be grown for a period of time sufficient to achieve a desired viable cell density of 1, 5, 10, 15, 20, 25, 30, 35, 40, 45, 50, 55, 60, 65, 70, 75, 80, 85, 90, 95 or 99 percent of maximal viable cell density.

In some embodiment, the cells are allowed to grow for a defined period of time. For example, depending on the starting concentration of the cell culture, the temperature at which the cells are grown, and the intrinsic growth rate of the cells, the cells may be grown for 0, 1, 2, 3, 4, 5, 6, 7, 8, 9, 10, 11, 12, 13, 14, 15, 16, 17, 18, 19, 20 or more days. In some cases, the cells may be allowed to grow for a month or more.

In some embodiments, the cells are allowed to grow to a desired viable cell density. For example, a desired viable cell density by the end of growth phase is greater than about 1.0×10⁶ viable cells/mL, 1.5×10⁶ viable cells/mL, 2.0×10⁶ viable cells/mL, 2.5×10⁶ viable cells/mL, 5×10⁶ viable cells/mL, 10×10⁶ viable cells/mL, 20×10⁶ viable cells/mL, 30×10⁶ viable cells/mL, 40×10⁶ viable cells/mL, or 50×10⁶ viable cells/mL.

The cell culture may be agitated or shaken during the initial culture phase in order to increase oxygenation and dispersion of nutrients to the cells. In accordance with the present invention, one of ordinary skill in the art will understand that it can be beneficial to control or regulate certain internal conditions of the bioreactor during the initial growth phase, including but not limited to pH, temperature, oxygenation, etc. For example, pH can be controlled by supplying an appropriate amount of acid or base and oxygenation can be controlled with sparging devices that are well known in the art. In some embodiments, a desired pH for the growth phase ranges from about 6.8-7.5 (e.g., about 6.9-7.4, about 6.9-7.3, about 6.95-7.3, about 6.95-7.25, about 7.0-7.3, about 7.0-7.25, about 7.0-7.2, about 7.0-7.15, about 7.05-7.3, about 7.05-7.25, about 7.05-7.15, about 7.05-7.20, about 7.10-7.3, about 7.10-7.25, about 7.10-7.20, about 7.10-7.15). In some embodiments, a desired pH for the growth phase is about 6.8, 6.85, 6.9, 6.95, 7.0, 7.05, 7.1, 7.15, 7.2, 7.25, 7.3, 7.35, 7.4, 7.45, or 7.5.

Transition Phase

In some embodiments, when the cells are ready for the production phase, the culture conditions may be changed to maximize the production of the recombinant protein of interest. Such culture condition change typically takes place in a transition phase. In some embodiments, such change may be a shift in one or more of a number of culture conditions including, but not limited to, temperature, pH, osmolarity and medium. In one embodiment, the pH of the culture is shifted. For example, the pH of the medium may be increased or decrease from growth phase to the production phase. In some embodiments, this change in pH is rapid. In some embodiments, this change in pH occurs slowly over a prolonged period of time. In some embodiments, the change in pH regulated by the addition of sodium biocarbonate. In some embodiments, the change in pH is initiated at the start of the transition phase and is maintained during the subsequent production phase.

In one embodiments, the glucose concentration of the cell culture medium is shifted. According to this embodiment, upon initiation of the transition phase, the glucose concentration within the cell culture is adjusted to a rate higher than 7.5 mM.

In some embodiments, the temperature is shifted up or down from the growth phase to production phase. For example, the temperature may be shifted up or down from growth phase to the production phase by about 0.1° C., 0.2° C., 0.3° C., 0.4° C., 0.5° C., 1.0° C., 1.5° C., 2.0° C., 2.5° C., 3.0° C., 3.5° C., 4.0° C., 4.5° C., 5.0° C., or more.

Production Phase

In accordance with the present invention, once the cell culture reaches a desired cell density and viability, with or without a transition phase, the cell culture is maintained for a subsequent production phase under culture conditions conducive to the survival and viability of the cell culture and appropriate for expression of I2S and/or FGE protein at commercially adequate levels.

In some embodiments, during the production phase, the culture is maintained at a temperature or temperature range that is lower than the temperature or temperature range of the growth phase. For example, during the production phase, cells may express recombinant I2S and/or FGE proteins well within a range of about 25° C. to 35° C. (e.g., about 28° C. to 35° C., about 30° C. to 35° C. about 32° C. to 35° C.). In some embodiments, during the production phase, cells may express recombinant I2S and/or FGE proteins well at a temperature of about 25° C., about 26° C., about 27° C., about 28° C., about 29° C., about 30° C., about 31° C., about 32° C., about 33° C., about 34° C., about 35° C., about 36° C., about 37° C. In other embodiments, during the production phase, the culture is maintained at a temperature or temperature range that is higher than the temperature or temperature range of the growth phase.

Additionally or alternatively, during the production phase, the culture is maintained at a pH or pH range that is different (lower or higher) than the pH or pH range of the growth phase. In some embodiments, the medium for the production phase has a pH ranging from about 6.8-7.5 (e.g., about 6.9-7.4, about 6.9-7.3, about 6.95-7.3, about 6.95-7.25, about 7.0-7.3, about 7.0-7.25, about 7.0-7.2, about 7.0-7.15, about 7.05-7.3, about 7.05-7.25, about 7.05-7.15, about 7.05-7.20, about 7.10-7.3, about 7.10-7.25, about 7.10-7.20, about 7.10-7.15). In some embodiments, the medium has a pH of about 6.8, 6.85, 6.9, 6.95, 7.0, 7.05, 7.1, 7.15, 7.2, 7.25, 7.3, 7.35, 7.4, 7.45, or 7.5.

In some embodiments, the cells may be maintained within a desired viable cell density range throughout the production. For example, during the production phase of the cell culture, a desired viable cell density may range from about 1.0-50×10⁶ viable cells/mL during the production phase (e.g., about 1.0-40×10⁶ viable cells/mL, about 1.0-30×10⁶ viable cells/mL, about 1.0-20×10⁶ viable cells/mL, about 1.0-10×10⁶ viable cells/mL, about 1.0-5×10⁶ viable cells/mL, about 1.0-4.5×10⁶ viable cells/mL, about 1.0-4×10⁶ viable cells/mL, about 1.0-3.5×10⁶ viable cells/mL, about 1.0-3×10⁶ viable cells/mL, about 1.0-2.5×10⁶ viable cells/mL, about 1.0-2.0×10⁶ viable cells/mL, about 1.0-1.5×10⁶ viable cells/mL, about 1.5-10×10⁶ viable cells/mL, about 1.5-5×10⁶ viable cells/mL, about 1.5-4.5×10⁶ viable cells/mL, about 1.5-4×10⁶ viable cells/mL, about 1.5-3.5×10⁶ viable cells/mL, about 1.5-3.0×10⁶ viable cells/mL, about 1.5-2.5×10⁶ viable cells/mL, about 1.5-2.0×10⁶ viable cells/mL).

In some embodiments, the cells may be maintained for a period of time sufficient to achieve a viable cell density of 1, 5, 10, 15, 20, 25, 30, 35, 40, 45, 50, 55, 60, 65, 70, 75, 80, 85, 90, 95 or 99 percent of maximal viable cell density. In some cases, it may be desirable to allow the viable cell density to reach a maximum. In some embodiments, it may be desirable to allow the viable cell density to reach a maximum and then allow the viable cell density to decline to some level before harvesting the culture. In some embodiments, the total viability at the end of the production phase is less than about 90%, 85%, 80%, 75%, 70%, 65%, 60%, 55%, 50%, 45%, 40%, 35%, 30%, 25%, 20%, 15%, 10%, 5%.

In some embodiments, the cells are allowed to grow for a defined period of time during the production phase. For example, depending on the concentration of the cell culture at the start of the subsequent growth phase, the temperature at which the cells are grown, and the intrinsic growth rate of the cells, the cells may be grown for about 5-90 days (e.g., about 5-80 days, about 5-70 days, about 5-60 days, about 5-50 days, about 5-40, about 5-30 days, about 5-20 days, about 5-15 days, about 5-10 days, about 10-90 days, about 10-80 days, about 10-70 days, about 10-60 days, about 10-50 days, about 10-40 days, about 10-30 days, about 10-20 days, about 15-90 days, about 15-80 days, about 15-70 days, about 15-60 days, about 15-50 days, about 15-40 days, about 15-30 days). In some embodiments, the production phase is lasted for about 5, 10, 15, 20, 25, 30, 35, 40, 45, 50, 55, 60, 65, 70, 75, 80, 85, or 90 days.

In some embodiments, the cells are maintained in the production phase until the titer to the recombinant I2S protein reaches a maximum. In other embodiments, the culture may be harvested prior to this point. For example, in some embodiments, the cells are maintained in the production phase until the titer to the recombinant I2S protein reaches a desired titer. Thus, a desired average harvest titer to the recombinant I2S protein may be of at least 6 mg per liter per day (mg/L/day) (e.g., at least 8, 10, 12, 14, 16, 18, 20, 25, 30, 35, 40, 45, 50, 55, 60, 65, 70, 75, 80, 85, 90, 95, 100, 150, 200, 250, 300, 350, 400, 450, or 500 mg/L/day, or more). In some embodiments, a desired average harvest titer to the recombinant I2S protein may range from about 6-500 mg/L/day (e.g., about 6-400 mg/L/day, about 6-300 mg/L/day, about 6-200 mg/L/day, about 6-100 mg/L/day, about 6-90 mg/L/day, about 6-80 mg/L/day, about 6-70 mg/L/day, about 6-60 mg/L/day, about 6-50 mg/L/day, about 6-40 mg/L/day, about 6-30 mg/L/day, about 10-500 mg/L/day, about 10-400 mg/L/day, about 10-300 mg/L/day, about 10-200 mg/L/day, about 10-100 mg/L/day, about 10-90 mg/L/day, about 10-80 mg/L/day, about 10-70 mg/L/day, about 10-60 mg/L/day, about 10-50 mg/L/day, about 10-40 mg/L/day, about 10-30 mg/L/day, about 20-500 mg/L/day, about 20-400 mg/L/day, about 20-300 mg/L/day, about 20-200 mg/L/day, about 20-100 mg/L/day, about 20-90 mg/L/day, about 20-80 mg/L/day, about 20-70 mg/L/day, about 20-60 mg/L/day, about 20-50 mg/L/day, about 20-40 mg/L/day, about 20-30 mg/L/day).

Additionally or alternatively, the cells are maintained in the production phase under conditions such that the produced recombinant I2S protein reach a desired C_{α}-formylglycine (FGly) conversion percentage. In some embodiments, the produced recombinant I2S protein contains at least about 70% (e.g., at least about 75%, 80%, 85%, 90%, 95%, 96%, 97%, 98%, 99%, 100%) conversion of the cysteine residue corresponding to Cys59 of human I2S protein to C_{α}-formylglycine (FGly).

Additionally or alternatively, the cells are maintained in the production phase under conditions such that the produced recombinant I2S protein reach a desired enzymatic activity. As can be appreciated by one skilled in the art, the enzymatic activity of recombinant I2S protein may be measured by various in vitro and in vivo assays. In some embodiments, a desired enzymatic activity, as measured by in vitro sulfate release activity assay using heparin disaccharide as substrate, of the produced recombinant I2S protein is at least about 20 U/mg, 30 U/mg, 40 U/mg, 50 U/mg, 60 U/mg, 70 U/mg, 80 U/mg, 90 U/mg, or 100 U/mg. In some embodiments, a desired enzymatic activity, as measured by in vitro sulfate release activity assay using heparin disaccharide as substrate, of the produced recombinant I2S protein ranges from about 20-100 U/mg (e.g., about 20-90 U/mg, about 20-80 U/mg, about 20-70 U/mg, about 20-60 U/mg, about 20-50 U/mg, about 20-40 U/mg, about 20-30 U/mg, about 30-100 U/mg, about 30-90 U/mg, about 30-80 U/mg, about 30-70 U/mg, about 30-60 U/mg, about 30-50 U/mg, about 30-40 U/mg, about 40-100 U/mg, about 40-90 U/mg, about 40-80 U/mg, about 40-70 U/mg, about 40-60 U/mg, about 40-50 U/mg). Exemplary conditions for performing in vitro sulfate release activity assay using heparin disaccharide as substrate are provided below. Typically, this assay measures the ability of I2S to release sulfate ions from a naturally derived substrate, heparin diasaccharide. The released sulfate may be quantified by ion chromatography. In some cases, ion chromatography is equipped with a conductivity detector. As a non-limiting example, samples are first buffer exchanged to 10 mM Na acetate, pH 6 to remove inhibition by phosphate ions in the formulation buffer. Samples are then diluted to 0.075 mg/ml with reaction buffer (10 mM Na acetate, pH 4.4) and incubated for 2 hrs at 37° C. with heparin disaccharide at an enzyme to substrate ratio of 0.3 μg I2S/100 μg substrate in a 30 μL reaction volume. The reaction is then stopped by heating the samples at 100° C. for 3 min. The analysis is carried out using a Dionex IonPac AS18 analytical column with an IonPac AG18 guard column. An isocratic method is used with 30 mM potassium hydroxide at 1.0 mL/min for 15 minutes. The amount of sulfate released by the I2S sample is calculated from the linear regression analysis of sulfate standards in the range of 1.7 to 16.0 nmoles. The reportable value is expressed as Units per mg protein, where 1 unit is defined as 1 μmoles of sulfate released per hour and the protein concentration is determined by A280 measurements.

In some embodiments, the enzymatic activity of recombinant I2S protein may also be determined using various other methods known in the art such as, for example, 4-MUF assay which measures hydrolysis of 4-methylumbelliferyl-sulfate to sulfate and naturally fluorescent 4-methylumbelliferone (4-MUF). In some embodiments, a desired enzymatic activity, as measured by in vitro 4-MUF assay, of the produced recombinant I2S protein is at least about 2 U/mg, 4 U/mg, 6 U/mg, 8 U/mg, 10 U/mg, 12 U/mg, 14 U/mg, 16 U/mg, 18 U/mg, or 20 U/mg. In some embodiments, a desired enzymatic activity, as measured by in vitro 4-MUF assay, of the produced recombinant I2S protein ranges from about 0-50 U/mg (e.g., about 0-40 U/mg, about 0-30 U/mg, about 0-20 U/mg, about 0-10 U/mg, about 2-50 U/mg, about 2-40 U/mg, about 2-30 U/mg, about 2-20 U/mg, about 2-10 U/mg, about 4-50 U/mg, about 4-40 U/mg, about 4-30 U/mg, about 4-20 U/mg, about 4-10 U/mg, about 6-50 U/mg, about 6-40 U/mg, about 6-30 U/mg, about 6-20 U/mg, about 6-10 U/mg). Exemplary conditions for performing in vitro 4-MUF assay are provided below. Typically, a 4-MUF assay measures the ability of an I2S protein to hydrolyze 4-methylumbelliferyl-sulfate (4-MUF-SO₄) to sulfate and naturally fluorescent 4-methylumbelliferone (4-MUF). One milliunit of activity is defined as the quantity of enzyme required to convert one nanomole of 4-MUF-SO₄ to 4-MUF in one minute at 37° C. Typically, the mean fluorescence units (MFU) generated by I2S test samples with known activity can be used to generate a standard curve, which can be used to calculate the enzymatic activity of a sample of interest.

In some embodiments, it may be beneficial or necessary to supplement the cell culture during the production phase with nutrients or other medium components that have been depleted or metabolized by the cells. For example, it might be advantageous to supplement the cell culture with nutrients or other medium components observed to have been depleted during the cell culture. Alternatively or additionally, it may be beneficial or necessary to supplement the cell culture prior to the production phase. As non-limiting examples, it may be beneficial or necessary to supplement the cell culture with redox-modulators, growth modulators (e.g., hormones and/or other growth factors), particular ions (such as sodium, chloride, calcium, magnesium, and phosphate), buffers, vitamins, nucleosides or nucleotides, trace elements (inorganic compounds usually present at very low final concentrations), amino acids, lipids, or glucose or other energy source.

These supplementary components may all be added to the cell culture at one time, or they may be provided to the cell culture in a series of additions. In some embodiments, the supplementary components are provided to the cell culture at multiple times in proportional amounts. In other embodiments, the cell culture is fed continually with these supplementary components. Typically, this process is known as perfusion and a cell culture involving perfusion is known as “perfusion culture.” As used herein, the term “perfusion culture” refers to a method of culturing cells in which additional components are provided continuously or semi-continuously to the culture subsequent to the beginning of the culture process. A portion of the cells and/or components in the medium are typically harvested on a continuous or semi-continuous basis and are optionally purified.

In some embodiments, the medium is continuously exchanged by a perfusion process during the production phase. Typically, volume of fresh medium relative to working volume of reactor per day (VVD) is defined as perfusion rate. Various perfusion rates may be used in according to the present invention. In some embodiments, a perfusion process has a perfusion rate such that the total volume added to the cell culture be kept to a minimal amount. In some embodiments, the perfusion process has a perfusion rate ranging from about 0.5-2 volume of fresh medium/working volume of reactor/day (VVD) (e.g., about 0.5-1.5 VVD, about 0.75-1.5 VVD, about 0.75-1.25 VVD, about 1.0-2.0 VVD, about 1.0-1.9 VVD, about 1.0-1.8 VVD, about 1.0-1.7 VVD, about 1.0-1.6 VVD, about 1.0-1.5 VVD, about 1.0-1.4 VVD, about 1.0-1.3 VVD, about 1.0-1.2 VVD, about 1.0-1.1 VVD). In some embodiments, the perfusion process has a perfusion rate of about 0.5, 0.55, 0.6, 0.65, 0.7, 0.75, 0.8, 0.85, 0.9, 0.95, 1.0, 1.05, 1.10, 1.15, 1.2, 1.25, 1.3, 1.35, 1.4, 1.45, 1.5, 1.55, 1.6, 1.65, 1.7, 1.75, 1.8, 1.85, 1.9, 1.95, or 2.0 VVD.

A perfusion process may also be characterized by volume of fresh medium added per cell per day, which is defined as cell specific perfusion rate. Various cell specific perfusion rates may be used. In some embodiments, the perfusion process has a cell specific perfusion rate ranging from about 0.05-5 nanoliter per cell per day (nL/cell/day) (e.g., about 0.05-4 mL/cell/day, about 0.05-3 mL/cell/day, about 0.05-2 mL/cell/day, about 0.05-1 mL/cell/day, about 0.1-5 mL/cell/day, about 0.1-4 mL/cell/day, about 0.1-3 mL/cell/day, about 0.1-2 mL/cell/day, about 0.1-1 mL/cell/day, about 0.15-5 mL/cell/day, about 0.15-4 mL/cell/day, about 0.15-3 mL/cell/day, about 0.15-2 mL/cell/day, about 0.15-1 mL/cell/day, about 0.2-5 mL/cell/day, about 0.2-4 mL/cell/day, about 0.2-3 mL/cell/day, about 0.2-2 mL/cell/day, about 0.2-1 mL/cell/day, about 0.25-5 mL/cell/day, about 0.25-4 mL/cell/day, about 0.25-3 mL/cell/day, about 0.25-2 mL/cell/day, about 0.25-1 mL/cell/day, about 0.3-5 mL/cell/day, about 0.3-4 mL/cell/day, about 0.3-3 mL/cell/day, about 0.3-2 mL/cell/day, about 0.3-1 mL/cell/day, about 0.35-5 mL/cell/day, about 0.35-4 mL/cell/day, about 0.35-3 mL/cell/day, about 0.35-2 mL/cell/day, about 0.35-1 mL/cell/day, about 0.4-5 mL/cell/day, about 0.4-4 mL/cell/day, about 0.4-3 mL/cell/day, about 0.4-2 nL/cell/day, about 0.4-1 mL/cell/day, about 0.45-5 mL/cell/day, about 0.45-4 mL/cell/day, about 0.45-3 mL/cell/day, about 0.45-2 mL/cell/day, about 0.45-1 mL/cell/day, about 0.5-5 mL/cell/day, about 0.5-4 mL/cell/day, about 0.5-3 mL/cell/day, about 0.5-2 mL/cell/day, about 0.5-1 mL/cell/day). In some embodiments, the perfusion process has a cell specific perfusion rate of about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mL/cell/day.

The cell culture may be agitated or shaken during the production phase in order to increase oxygenation and dispersion of nutrients to the cells. In accordance with the present invention, one of ordinary skill in the art will understand that it can be beneficial to control or regulate certain internal conditions of the bioreactor during the growth phase, including but not limited to pH, temperature, oxygenation, etc. For example, pH can be controlled by supplying an appropriate amount of acid or base and oxygenation can be controlled with sparging devices that are well known in the art. One or more antiform agents may also be provided.

Same culture medium may be used throughout the production process including the growth phase, production phase and profusion. In some embodiments, at least two different media are used in the production of recombinant I2S. For example, a nutrient medium formulated for cell growth is often used to support growth of the cells throughout the cell growth phase, and nutrient medium formulated for protein production is used during the production phase of the process to support expression and harvesting of I2S. In either case, the nutrient medium may or may not contain serum or other animal-derived components (e.g., fetuin).

According to the present invention, the cells are typically grown in suspension. However, the cells may be attached to a substrate. In one example, cells may be attached to microbead or particles which are suspended in the nutrient medium.

Bioreactors

The invention also provides bioreactors that are useful for producing recombinant iduronate-2-sulfatase. Bioreactors may be perfusion, batch, fed-batch, repeated batch, or continuous (e.g. a continuous stirred-tank reactor models), for example. Typically, the bioreactors comprise at least one vessel designed and are configured to house medium (e.g., a chemically defined nutrient medium). The vessel also typically comprises at least one inlet designed and configured to flow fresh nutrient medium into the vessel. The vessel also typically comprises at least one outlet designed and configured to flow waste medium out of the vessel. In some embodiments, the vessel may further comprise at least one filter designed and configured to minimize the extent to which isolated cells in the vessel are passed out through the at least one outlet with waste medium. The bioreactor may also be fitted with one or more other components designed to maintain conditions suitable for cell growth. For example, the bioreactor may be fitted with one or more circulation or mixing devices designed and configured to circulate or mix the nutrient medium within the vessel. Typically, the isolated cells that are engineered to express recombinant I2S are suspended in the nutrient medium. Therefore, in some cases, the circulation device ensures that the isolated cells remain in suspension in the nutrient medium. In some cases, the cells are attached to a substrate. In some cases, the cells are attached to one or more substrates (e.g., microbeads) that are suspended in the nutrient medium. The bioreactor may comprise one or more ports for obtaining a sample of the cell suspension from the vessel. The bioreactor may be configured with one or more components for monitoring and/or controlling conditions of the culture, including conditions such as gas content (e.g., air, oxygen, nitrogen, carbon dioxide), flow rates, temperature, pH and dissolved oxygen levels, and agitation speed/circulation rate.

Vessels of any appropriate size may be used in the bioreactors. Typically, the vessel size is suitable for satisfying the production demands of manufacturing recombinant I2S. In some embodiments, the vessel is designed and configured to contain up to 1 L, up to 10 L, up to 100 L, up to 500 L, up to 1000 L, up to 1500 L, up to 2000 L, or more of the nutrient medium. In some embodiments, the volume of the production bioreactor is at least 10 L, at least 50 L, 100 L, at least 200 L, at least 250 L, at least 500 L, at least 1000 L, at least 1500 L, at least 2000 L, at least 2500 L, at least 5000 L, at least 8000 L, at least 10,000 L, or at least 12,000 L, or more, or any volume in between. The production bioreactor may be constructed of any material that is conducive to cell growth and viability that does not interfere with expression or stability or activity of the produced I2S protein. Exemplary material may include, but not be limited to, glass, plastic, or metal.

In some embodiments, cells may be cultured in a chemically defined medium that is housed in a vessel of a bioreactor. The culture methods often involve perfusing fresh nutrient medium into the vessel through the at least one inlet and bleeding waste nutrient medium out from vessel through the at least one outlet. Bleeding is performed at a rate of up to about 0.1 vessel volume per day, about 0.2 vessel volume per day, about 0.3 vessel volume per day, about 0.4 vessel volume per day, about 0.5 vessel volume per day, about 1 vessel volume per day, about 1.5 vessel volumes per day or more. The methods also involve harvesting nutrient medium that comprises recombinant I2Ss. Harvesting may be performed at a rate of up to about 0.1 vessel volume per day, about 0.2 vessel volume per day, about 0.3 vessel volume per day, about 0.4 vessel volume per day, about 0.5 vessel volume per day, about 1 vessel volume per day, about 1.5 vessel volumes per day or more. Perfusing is also performed, typically at a rate equivalent to the sum of the bleeding rate and the harvesting rate. For example, perfusion rate may be great than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 vessel volume per day. In some embodiments, perfusion rate is less than about 5.0, 4.5, 4.0, 3.5, 3.0, 2.5, 2.0, 1.5, 1.4, 1.3, 1.2, 1.1, 1.0, 0.9, 0.8, 0.7, 0.6, 0.5 vessel volume per day. Exemplary perfusion rates are described throughout the specification.

Monitoring Culture Conditions

In certain embodiments of the present invention, the practitioner may find it beneficial or necessary to periodically monitor particular conditions of the growing cell culture. Monitoring cell culture conditions allows the practitioner to determine whether the cell culture is producing recombinant polypeptide or protein at suboptimal levels or whether the culture is about to enter into a suboptimal production phase. In order to monitor certain cell culture conditions, it will be necessary to remove small aliquots of the culture for analysis.

As non-limiting example, it may be beneficial or necessary to monitor temperature, pH, cell density, cell viability, integrated viable cell density, osmolarity, or titer or activity of the expressed I2S protein. Numerous techniques are well known in the art that will allow one of ordinary skill in the art to measure these conditions. For example, cell density may be measured using a hemacytometer, a Coulter counter, or Cell density examination (CEDEX). Viable cell density may be determined by staining a culture sample with Trypan blue. Since only dead cells take up the Trypan blue, viable cell density can be determined by counting the total number of cells, dividing the number of cells that take up the dye by the total number of cells, and taking the reciprocal. Alternatively, the level of the expressed I2S protein can be determined by standard molecular biology techniques such as coomassie staining of SDS-PAGE gels, Western blotting, Bradford assays, Lowry assays, Biuret assays, and UV absorbance. It may also be beneficial or necessary to monitor the post-translational modifications of the expressed I2S protein, including phosphorylation and glycosylation.

### Purification of Expressed I2S Protein

Various methods may be used to purify or isolate I2S protein produced according to various methods described herein. In some embodiments, the expressed I2S protein is secreted into the medium and thus cells and other solids may be removed, as by centrifugation or filtering for example, as a first step in the purification process. Alternatively or additionally, the expressed I2S protein is bound to the surface of the host cell. In this embodiment, the host cells (for example, yeast cells) expressing the polypeptide or protein are lysed for purification. Lysis of host cells (e.g., yeast cells) can be achieved by any number of means well known to those of ordinary skill in the art, including physical disruption by glass beads and exposure to high pH conditions.

The I2S protein may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly desired when cells must be lysed in order to isolate and purify the expressed polypeptide or protein.

Exemplary purification methods are described in the Examples sections below. Additional purification methods are described in the provisional application entitled “Purification of Recombinant I2S Protein” filed on herewith on even date, the entire disclosure of which is hereby incorporated by reference.

### Pharmaceutical Composition and Administration

Purified recombinant I2S protein may be administered to a Hunter Syndrome patient in accordance with known methods. For example, purified recombinant I2S protein may be delivered intravenously, subcutaneously, intramuscularly, parenterally, transdermally, or transmucosally (e.g., orally or nasally)).

In some embodiments, a recombinant I2S or a pharmaceutical composition containing the same is administered to a subject by intravenous administration.

In some embodiments, a recombinant I2S or a pharmaceutical composition containing the same is administered to a subject by intrathecal administration. As used herein, the term “intrathecal administration” or “intrathecal injection” refers to an injection into the spinal canal (intrathecal space surrounding the spinal cord). Various techniques may be used including, without limitation, lateral cerebroventricular injection through a burrhole or cisternal or lumbar puncture or the like. In some embodiments, “intrathecal administration” or “intrathecal delivery” according to the present invention refers to IT administration or delivery via the lumbar area or region, i.e., lumbar IT administration or delivery. As used herein, the term “lumbar region” or “lumbar area” refers to the area between the third and fourth lumbar (lower back) vertebrae and, more inclusively, the L2-S 1 region of the spine.

In some embodiments, a recombinant I2S or a pharmaceutical composition containing the same is administered to the subject by subcutaneous (i.e., beneath the skin) administration. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems.

In some embodiments, intrathecal administration may be used in conjunction with other routes of administration (e.g., intravenous, subcutaneously, intramuscularly, parenterally, transdermally, or transmucosally (e.g., orally or nasally)).

The present invention contemplates single as well as multiple administrations of a therapeutically effective amount of a recombinant I2S or a pharmaceutical composition containing the same described herein. A recombinant I2S or a pharmaceutical composition containing the same can be administered at regular intervals, depending on the nature, severity and extent of the subject's condition (e.g., a lysosomal storage disease). In some embodiments, a therapeutically effective amount of a recombinant I2S or a pharmaceutical composition containing the same may be administered periodically at regular intervals (e.g., once every year, once every six months, once every five months, once every three months, bimonthly (once every two months), monthly (once every month), biweekly (once every two weeks), weekly, daily or continuously).

A recombinant I2S or a pharmaceutical composition containing the same can be formulated with a physiologically acceptable carrier or excipient to prepare a pharmaceutical composition. The carrier and therapeutic agent can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

As used herein, the term “therapeutically effective amount” is largely determined base on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition). For example, a therapeutically effective amount may be an amount sufficient to achieve a desired therapeutic and/or prophylactic effect, such as an amount sufficient to modulate lysosomal enzyme receptors or their activity to thereby treat such lysosomal storage disease or the symptoms thereof (e.g., a reduction in or elimination of the presence or incidence of “zebra bodies” or cellular vacuolization following the administration of the compositions of the present invention to a subject). Generally, the amount of a therapeutic agent (e.g., a recombinant lysosomal enzyme) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Additional exemplary pharmaceutical compositions and administration methods are described in PCT Publication WO2011/163649 entitled “Methods and Compositions for CNS Delivery of Iduronate-2-Sulfatase;” and provisional application Ser. No. 61/618,638 entitled “Subcutaneous administration of iduronate 2 sulfatase” filed on Mar. 30, 2012, the entire disclosures of both of which are hereby incorporated by reference.

It is to be further understood that for any particular subject, specific dosage regimens should be adjusted over time according to the individual need and the professional judgment of the person administering or supervising the administration of the enzyme replacement therapy and that dosage ranges set forth herein are exemplary only and are not intended to limit the scope or practice of the claimed invention.

### EXAMPLES

### Example 1

### Generation of Optimized Cell Line Co-Expressing Recombinant I2S and FGE

This example illustrates an exemplary cell line co-expressing recombinant I2S and FGE that can be used to produce recombinant I2S protein. It will be clear to one skilled in the art, that a number of alternative approaches, expression vectors and cloning techniques are available.

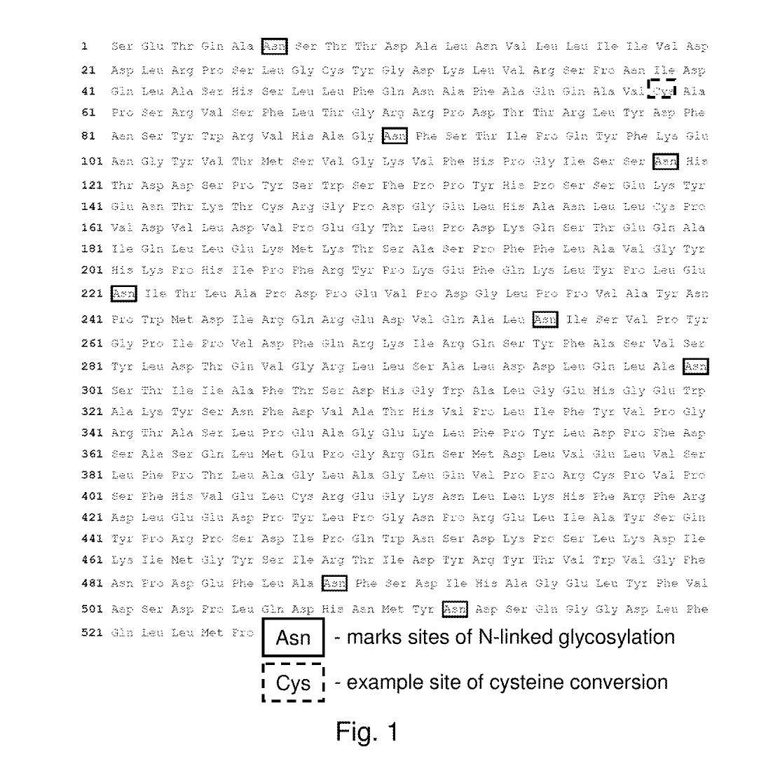

A typical mature form of human iduronate-2-sulfatase enzyme (I2S) is a 525-amino acid glycoprotein that undergoes extensive processing and post translational modification for enzyme activation, such as glycosylation and cysteine conversion to formylgycine (FIG. 1). In mammalian cells, conserved cysteine residues within the I2S (i.e., at amino acid 59) enzyme are converted to formylglycine by the formylglycine generating enzyme (FGE). The conversion of cysteine to formylglycine within the active site of the I2S enzyme is an important step in generating the active form of the human sulfatase enzyme. The purpose of this experiment was to engineer an optimized human cell line co-expressing I2S and FEG for generating active recombinant I2S.

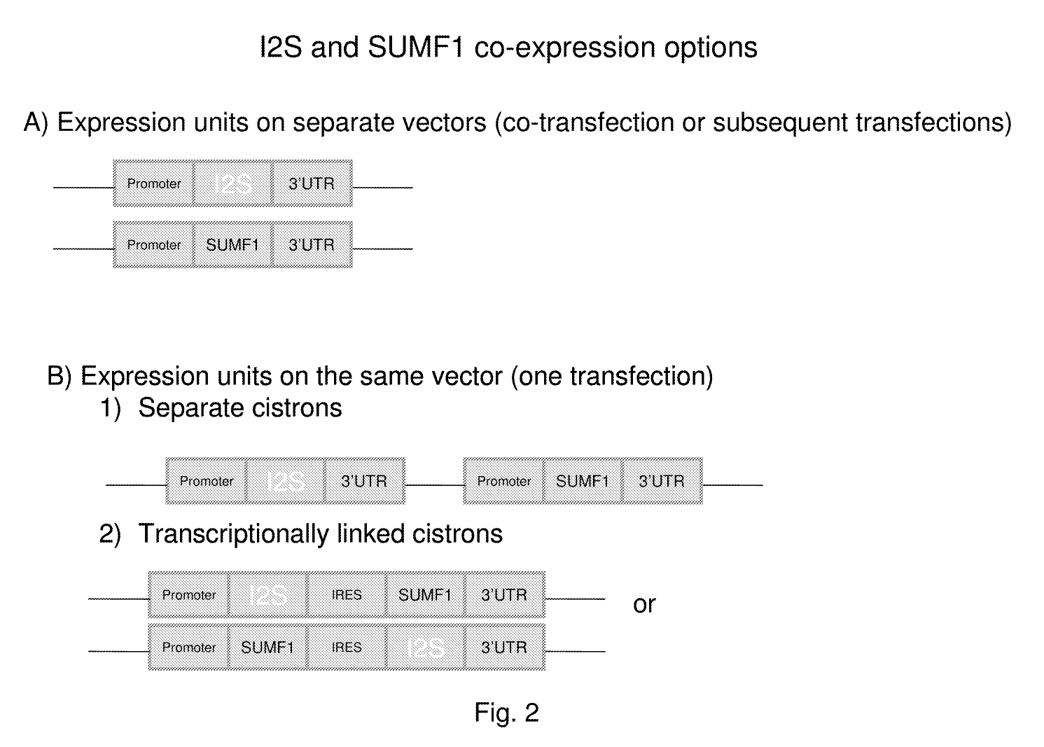

FIG. 2 illustrates a number of exemplary construct designs for co-expression of I2S and FGE. For example, expression units of I2S and FGE can be located on separate vectors and the separate vectors can be co-transfected or transfected separately (FIG. 2A). Alternatively, expression units of I2S and FGE can be located on the same vector (FIG. 2B). In one configuration, I2S and FGE can be on the same vector but under the control of separate promoters, also referred to as separate cistrons (FIG. 2B(1)). Alternatively, I2S and FGE can be designed as transcriptionally linked cistrons, that is, I2S and FGE are designed as one open reading frame under the control of a same promoter (FIG. 2B(2)). Typically, an internal ribosome entry site (IRES) is designed to allow translation initiation in the middle of the messenger RNA (mRNA) (FIG. 2B(2)).

A human cell line was engineered to co-express human I2S protein with the amino acid sequence shown in SEQ ID NO:2 and human formylglycine generating enzyme (FGE) with the amino acid sequence shown in SEQ ID NO:6.

>Full-lenth Precursor iduronate 2-sulfatase
SEQ ID NO: 2
MPPPRTGRGLLWLGLVLSSVCVALGSETQANSTTDALNVLLIIVDDLR
PSLGCYGDKLVRSPNIDQLASHSLLFQNAFAQQAVCAPSRVSFLTGRR
PDTTRLYDFNSYWRVHAGNFSTIPQYFKENGYVTMSVGKVFHPGISSN
HTDDSPYSWSFPPYHPSSEKYENTKTCRGPDGELHANLLCPVDVLDVP
EGTLPDKQSTEQAIQLLEKMKTSASPFFLAVGYHKPHIPFRYPKEFQK
LYPLENITLAPDPEVPDGLPPVAYNPWMDIRQREDVQALNISVPYGPI
PVDFQRKIRQSYFASVSYLDTQVGRLLSALDDLQLANSTIIAFTSDHG
WALGEHGEWAKYSNFDVATHVPLIFYVPGRTASLPEAGEKLFPYLDPF
DSASQLMEPGRQSMDLVELVSLFPTLAGLAGLQVPPRCPVPSFHVELC
REGKNLLKHFRFRDLEEDPYLPGNPRELIAYSQYPRPSDIPQWNSDKP
SLKDIKIMGYSIRTIDYRYTVWVGFNPDEFLANFSDIHAGELYFVDSD
PLQDHNMYNDSQGGDLFQLLMP
Full-length human FGE precursor:
SEQ ID NO: 6
MAAPALGLVCGRCPELGLVLLLLLLSLLCGAAGSQEAGTGAGAGSLAG
SCGCGTPQRPGAHGSSAAAHRYSREANAPGPVPGERQLAHSKMVPIPA
GVFTMGTDDPQIKQDGEAPARRVTIDAFYMDAYEVSNTEFEKFVNSTG
YLTEAEKFGDSFVFEGMLSEQVKTNIQQAVAAAPWWLPVKGANWRHPE
GPDSTILHRPDHPVLHVSWNDAVAYCTWAGKRLPTEAEWEYSCRGGLH
NRLFPWGNKLQPKGQHYANIWQGEFPVTNTGEDGFQGTAPVDAFPPNG
YGLYNIVGNAWEWTSDWWTVHHSVEETLNPKGPPSGKDRVKKGGSYMC
HRSYCYRYRCAARSQNTPDSSASNLGFRCAADRLPTMD

Both I2S and FGE expression are controlled by a human CMV promoter. Translation of I2S mRNA results in synthesis of a 550 amino acid full length I2S protein (SEQ ID NO:2), which includes a 25 amino acid signal peptide. The signal peptide is removed and a soluble enzyme is secreted from the cell.

The bacterial neomycin phosphotransferase (neo) coding sequence and/or Blasticidin S Deaminase (BSD) gene were used to allow for selection of transfected cells using the neomycin analog G418 and/or blasticidin, respectively. In addition, the mouse dihydrofolate reductase (DHFR) gene was used on the I2S- and/or FGE-encoding vector(s) to allow for isolation of cell lines containing increased copies of the I2S- and/or FGE-encoding sequences by methotrexate (MTX) selection.

Cells producing I2S were isolated and subjected to appropriate drug selection to isolate cells with an increased number of copies of the transfected I2S and/or FGE genes. Quantification of I2S was performed by ELISA.

The cell population was also subjected to step-wise selection in methotrexate (MTX) to isolate cells with increased I2S productivity. I2S productivity was monitored during MTX selection by ELISA.

After several rounds of propagation, several I2S producing clones were then subjected to suspension adaptation in serum-free media through a stepwise reduction from DMEM containing 10% calf serum to serum free chemically defined media. Several individual clonal populations were established through limited dilution cloning. Colonies were screened by I2S enzyme activity assay and ELISA. Two stable cell lines 2D and 4D showed high percent viability and robust expression of I2S and were selected for further development.

### Example 2

### Serum-Free Suspension Cell Culture

This example demonstrates that a serum-free cell culture system may be used to successfully cultivate a cell line co-expressing I2S and FGE to produce recombinant I2S.

### Generating a Seed Culture

Briefly, a seed culture was established using the 2D or 4D cell lines of Example 1. Cells were transferred to a 250 ml vented tissue culture shake flask containing serum-free chemically defined expansion medium, supplemented with Methotrexate for selection, adjusted with sodium bicarbonate to a pH of 7.3 and grown under standard conditions.

### Cell Culture Expansion

Upon reaching the desired viable cell density, the initial seed culture was used to inoculate the first of a series of step-wise cell culture expansions consisting of a 500 ml tissue culture shake flask followed by 2×1 L tissue culture shake flasks. In each case, the preceding cell culture was transferred in its entirety to inoculate the subsequent larger culture flask, upon reaching a desired cell density.

A batch culture expansion was performed by transferring each of the 2×1 L cultures into a 10 L Cellbag Bioreactor® (Wave Europe), and adding expansion medium to a final weight of 2.5 kg. After reaching a desired cell density, new expansion medium was added to a final weight of 5.0 kg and the cells grown to a desired density. The 10 L Cellbag was transferred to a Wave Bioreactor® system (Wave Europe) and culture conditions were modified to allow for growth under continuous medium perfusion. Expansion growth medium was delivered at a target weight of 5.0 L per day (1.0 vvd) and samples were collected for off-line metabolite analysis of pH, glutamine, glutamate, glucose, ammonium, lactate, pCO₂ and osmolarity.

Upon reaching a desired cell density, the entire 10 L cell culture was transferred to a 50 L Wave Cellbag Bioreactor®, containing 20 kg of fresh expansion medium, and again grown to a desired cell density.

### Bioreactor Expansion

Cell expansion was next performed using a 200 L disposable bioreactor and centrifuge perfusion device (Centritech® CELL II unit, Pneumatic Scale Corporation), which is designed to concentrate cells and clarify media for recycling during perfusion mediated cell culture. Expansion medium was inoculated with a portion of the 50 L culture sufficient to achieve a desired cell density.

Next a portion of the 200 L culture was used to seed a 2000 L disposable bioreactor and centrifuge perfusion device (Centritech® CELL II unit, Pneumatic Scale Corporation). Cells were grown under batch growth conditions for two days. Following the two day growth, conditions were adjusted for continuous perfusion, initiating the start of the transition phase.

### Bioreactor Production

For the production phase, two Centritech CELL II units were used. Production phase was started approximately 24 hours after the start of the transition phase, at which time the cells typically had achieved a desired cell density. Cell density was maintained for a desired production period, by regulating the bleed rate.

### Example 3

### Physiochemical and Biological Characterization of Recombinant I2S Enzyme Produced in Serum-Free Cell Culture

The purpose of the example was to perform a detailed characterization of the recombinant I2S protein produced using the serum-free cell culture method described above.

### SDS-PAGE

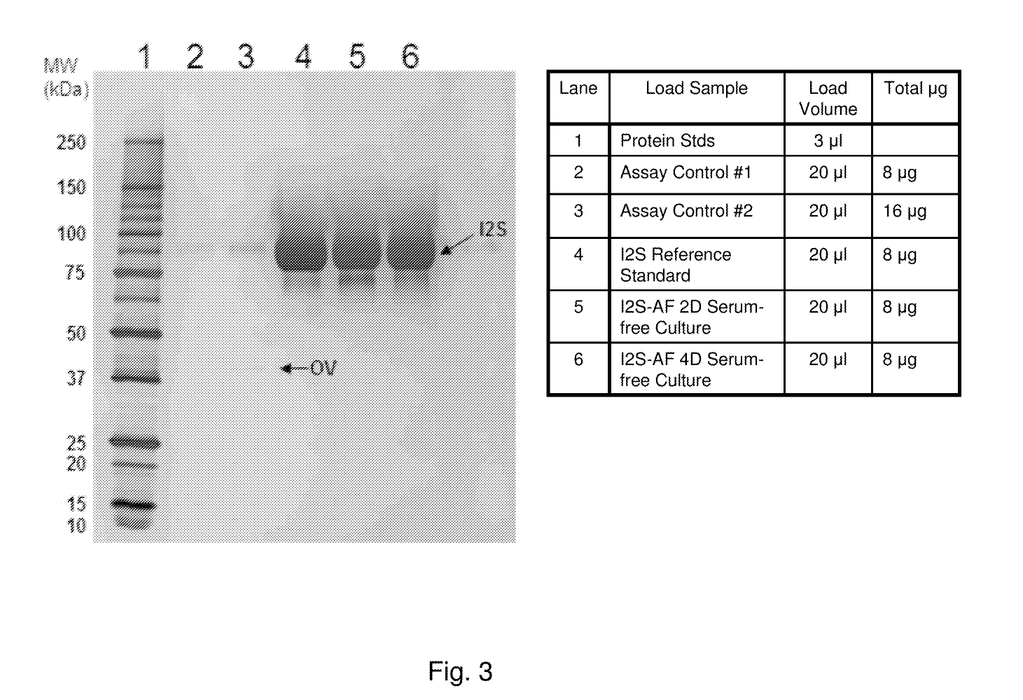

For this experiment, recombinant I2S protein was generated using the 2D and 4D human cell lines, in two separate serum-free cell culture reactions using the methods described above. Samples were collected during the Production Phase, and the purified I2S enzyme was analyzed by SDS-PAGE, and treated with silver stain for visualization. FIG. 3 shows, that in each of the separate manufacturing experiments, I2S protein produced from the 2D and 4D cell lines under serum-free conditions migrated at the appropriate size (Lanes 5 and 6), as indicated upon comparison with the molecular weight protein standard (Lane 1) and commercially available I2S assay controls (Lanes 2 and 3). Furthermore, the recombinant I2S produced under the serum-free condition (Lanes 5 and 6) also migrated at the same size as I2S Reference Standard (Lane 4).

### Peptide Map

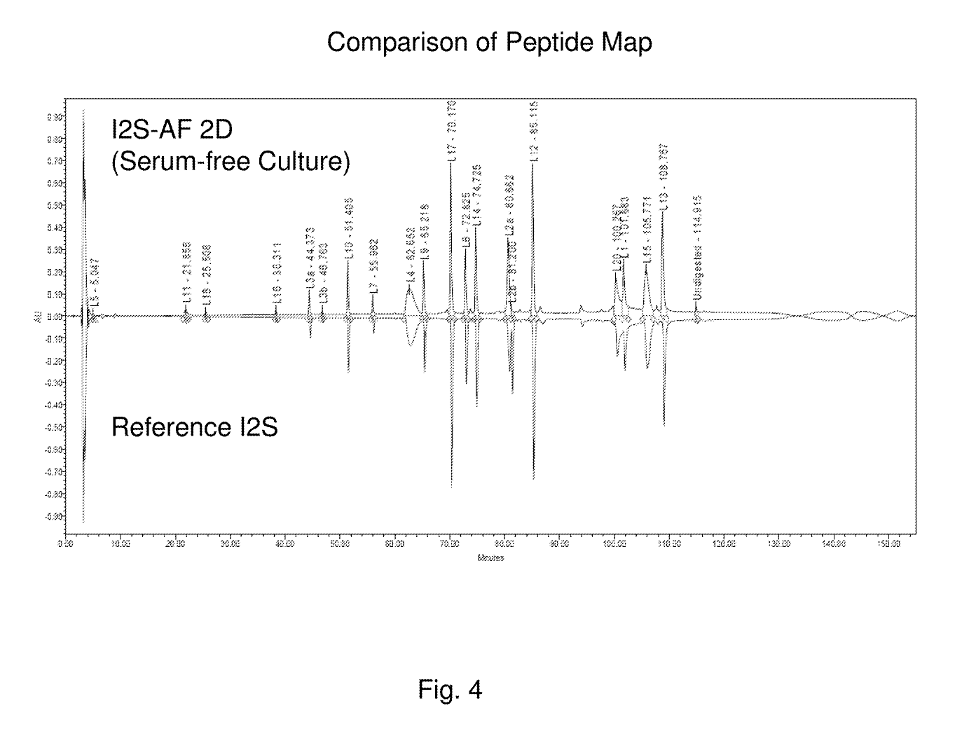

Recombinant I2S protein was generated using the I2S-AF 2D cell line grown under the serum-free culture conditions described above. The isolated recombinant I2S generated from the I2S-AF 2D cell line and a sample of reference human I2S were each subjected to proteolytic digest (e.g., by trypsin) and examined by HPLC analysis. Exemplary results are shown in FIG. 4.

### Percent Formylglycine Conversion

Peptide mapping can be used to determine Percent FGly conversion. I2S activation requires Cysteine (corresponding to position 59 of mature human I2S) to formylglycine conversion by formylglycine generating enzyme (FGE) as shown below:

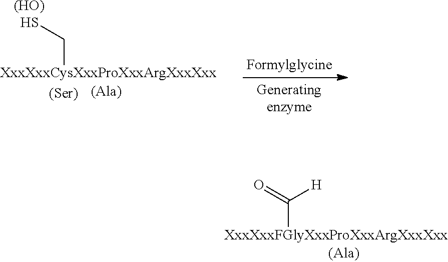

Therefore, the percentage of formylglycine conversion (% FG) can be calculated using the following formula:

$$\% FG ( of DS ) = \frac{Number of active I 2 S molecules}{Number of total ( active + inactive ) I 2 S molecules} \times 100$$

For example 50% FG means half of the purified recombinant I2S is enzymatically inactive without any therapeutic effect.

Peptide mapping was used to calculate % FG. Briefly, a recombinant I2S protein was digested into short peptides using a protease (e.g., trypsin or chymotrypsin). Short peptides were separated and characterized using HPLC. The peptide containing the position corresponding to position 59 of the mature human I2S was characterized to determine if the Cys at position 59 was converted to a FGly as compared to a control (e.g., an I2S protein without FGly conversion or an I2S protein with 100% FGly conversion). The amount of peptides containing FGly (corresponding to number of active I2S molecules) and the total amount of peptides with both FGly and Cys (corresponding to number of total I2S molecules) may be determined based on the corresponding peak areas and the ratio reflecting % FG was calculated. Exemplary results are shown in Table 4.

### Glycan Map—Mannose-6-Phosphate and Sialic Acid Content

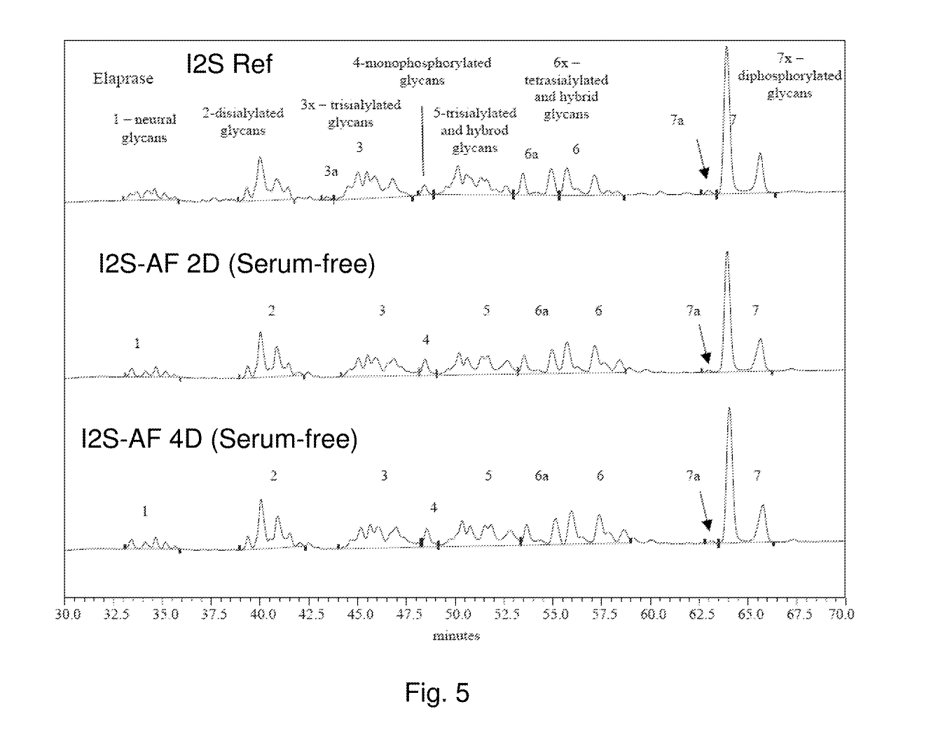

The glycan and sialic acid composition of recombinant I2S protein produced under serum-free cell culture conditions was determined. Quantification of the glycan composition was performed, using anion exchange chromatography. As described below, the glycan map of recombinant I2S generated under these conditions consists of seven peak groups, eluting according to an increasing amount of negative charges, at least partly derived from sialic acid and mannose-6-phosphate glycoforms resulting from enzymatic digest. Briefly, purified recombinant I2S obtained using the serum-free cell culture method (I2S-AF 2D Serum-free and I2S-AF 4D Serum-free) and reference recombinant I2S produced, were treated with either (1) purified neuraminidase enzyme (isolated from Arthrobacter Ureafaciens (10 mU/μL), Roche Biochemical (Indianapolis, Ind.), Cat. #269 611 (1U/100 μL)) for the removal of sialic acid residues, (2) alkaline phosphatase for 2 hours at 37±1° C. for complete release of mannose-6-phosphate residues, (3) alkaline phosphatase+neuraminidase, or (4) no treatment. Each enzymatic digest was analyzed by High Performance Anion Exchange Chromatography with Pulsed Amperometric Detection (HPAE-PAD) using a CarboPac PA1 Analytical Column equipped with a Dionex CarboPac PA1 Guard Column. A series of sialic acid and mannose-6-phosphate standards in the range of 0.4 to 2.0 nmoles were run for each assay. An isocratic method using 48 mM sodium acetate in 100 mM sodium hydroxide was run for a minimum of 15 minutes at a flow rate of 1.0 mL/min at ambient column temperature to elute each peak. The data generated from each individual run, for both the I2S-AF and reference I2S samples, were each combined into a single chromatograph to represent the glycan map for each respective recombinant protein. As indicated in FIG. 5, an exemplary glycan map for I2S produced using the human cell serum-free method displayed representative elution peaks (in the order of elution) constituting neutrals, mono-, disialyated, monophosphorylated, trisialyated and hybrid (monosialyated and capped mannose-6-phosphate), tetrasialylated and hybrid (disilaylated and capped mannose-6-phosphate) and diphosphorylated glycans.

Average sialic acid content (moles sialic acid per mole protein) in each recombinant I2S sample was calculated from linear regression analysis of sialic acid standards. Each chromatogram run was visualize using the PeakNet 6 Software. Sialic acid standards and sialic acid released from recombinant I2S assay control and test samples appear as a single peak. The amount of sialic acid (nmoles) for I2S was calculated as a raw value using the following equation:

$$S . A . ( mole per mole I 2 S ) = \frac{( nmoles sialic acid )}{( 0.3272 ) ( C )}$$

Where C is the protein concentration (in mg/ml) of sample or recombinant I2S assay control. The corrected value of sialic acid as moles of sialic acid per mole of protein for each test sample was calculated using the following formula:

$$Corrected S . A . = \frac{\begin{matrix}( Sample Raw Sialic Acid Value ) \times \\ ( Established Idursulfase Assay Control Value )\end{matrix}}{( Idursulfase Assay Control Raw Sialic Acid Value )}$$

Exemplary data indicative of sialic acid content on the recombinant I2S produced by I2S-AF 2D or 4D cell lines are shown in Table 4.

TABLE 4
Exemplary Characteristics of Recombinant 12S Produced in Serum-Free
Cell Culture

| | |
|---|---|
| | I2S-AF 2D |
| Assay | (Serum-free) |

Peptide Mapping

| | |
|---|---|
| L1 | 101 |
| L10 | 100 |
| L12 | 102 |
| L13 | 97 |
| L14 | 101 |
| L17 | 100 |
| L20 | 102 |
| Host Cell Protein | <62.5 ng/mg |

Ion Exchange HPLC % Area

| | |
|---|---|
| Peak A | 62 |
| Peak A + B | 82 |
| Peak E + F | 0 |
| % Formylglycine | 87 |
| Specific activity (U/mg) | 64 |
| (sulfate release assay) | |
| % Size Exclusion | ≧99.8 |
| HPLC | |

Glycan Mapping

| | |
|---|---|
| Monosialylated | 105 |
| Disialylated | 93 |
| Monophosphorylated | 139 |
| Trisialylated | 89 |
| Tetrasialylated | 125 |
| Diphosphorylated | 95 |
| Sialic Acid (mol/mol) | 20 |

### Specific Activity

Specific activity of the recombinant I2S enzyme produced using the 2D and 4D cell lines under serum-free cell culture conditions was analyzed using in vitro sulfate release assay or 4-MUF assay.

In Vitro Sulfate Release Assay

In vitro sulfate release activity assay was conducted using heparin disaccharide as substrate. In particular, this assay measures the ability of I2S to release sulfate ions from a naturally derived substrate, heparin diasaccharide. The released sulfate may be quantified by ion chromatography equipped with a conductivity detector. Briefly, samples were first buffer exchanged to 10 mM Na acetate, pH 6 to remove inhibition by phosphate ions in the formulation buffer. Samples were then diluted to 0.075 mg/ml with reaction buffer (10 mM Na acetate, pH 4.4) and incubated for 2 hrs at 37° C. with heparin disaccharide at an enzyme to substrate ratio of 0.3 μg I2S/100 μg substrate in a 30 μL reaction volume. The reaction was then stopped by heating the samples at 100° C. for 3 min. The analysis was carried out using a Dionex IonPac AS18 analytical column with an IonPac AG18 guard column. An isocratic method was used with 30 mM potassium hydroxide at 1.0 mL/min for 15 minutes. The amount of sulfate released by the I2S sample was calculated from the linear regression analysis of sulfate standards in the range of 1.7 to 16.0 nmoles. The reportable value was expressed as Units per mg protein, where 1 unit is defined as 1 μmoles of sulfate released per hour and the protein concentration is determined by A280 measurements. Exemplary results are shown in Table 4.

4-MUF Assay

Specific activity of the recombinant I2S enzyme produced using the 2D and 4D cell lines under serum-free cell culture conditions may also be analyzed using the fluorescence based 4-MUF assay. Briefly, the assay measures the hydrolysis of I2S substrate 4-methylumbelliferyl-sulfate (4-MUF-SO₄). Upon cleavage of the 4-MUF-SO₄ substrate by I2S, the molecule is converted to sulfate and naturally fluorescent 4-methylumbelliferone (4-MUF). As a result, I2S enzyme activity can be determined by evaluating the overall change in fluorescent signal over time. For this experiment, purified I2S enzyme produced from the I2S-AF 2D and 4D human cell lines were incubated with a solution of 4-methylumbelliferyl-sulfate (4-MUF-SO₄), Potassium Salt, Sigma Cat. #M-7133). Calibration of the assay was performed using a series of control reference samples, using commercially available I2S enzyme diluted at 1:100, 1:200 and 1:20,000 of the stock solution. The enzymatic assay was run at 37° C. and assayed using a calibrated fluorometer. Using the fluorescence values obtained for each reference standard, the percent coefficient of variation was determined using the following equation:

$$\% CV = \frac{Standard Deviation of Raw Fluorescenc Values ( N = 3 )}{Averagel Fluorescence Value} \times 100 \%$$

The percent CV values were then used to calculate the Corrected Average Fluorescence for each sample, in order to determine the reportable enzyme activity, expressed in mU/mL using the following formula:

$$mU / mL = ( CFU ) ( \frac{1 nmole / L}{10 FU} ) ( \frac{1 L}{{10}^{3} mL} ) ( \frac{2.11 mL}{0.01 mL} ) ( \frac{1 hour}{60 min} ) ( \frac{1 mU}{nmole} ) ( DF )$$

CFU=Negative corrected average fluorescence

DF—Dilution Factor

One milliunit of activity is the quantity of enzyme required to convert 1 nanomole of 4-methylumbelliferyl-sulfate to 4-methylumbelliferone in 1 minute at 37° C.

### Charge Profile

The charge distribution of each purified recombinant I2S was determined by Strong Anion Exchange (SAX) Chromatography, with a High Performance Liquid Chromatography (HPLC) system. The method separates recombinant I2S variants within the sample, based on surface charge differences. At pH 8.00, negatively charged species adsorb onto the fixed positive charge of the SAX column. A gradient of increasing ionic strength is used to elute each protein species in proportion to the strength of their ionic interaction with the column. One hundred micrograms of purified I2S, isolated from the 2D cell line under serum-free growth conditions or reference recombinant I2S enzyme, was loaded onto an Amersham Biosciences

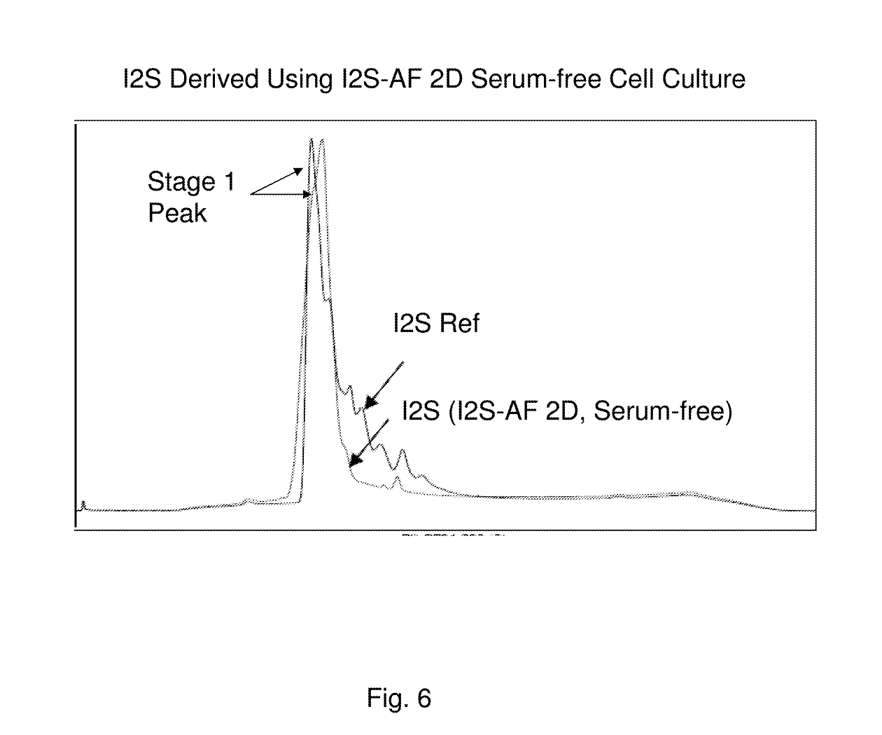

Mini Q PE (4.6×50 mm) column held at ambient temperature and equilibrated to 20 mM Tris-HCl, pH 8.00. Gradient elution was made at a flow rate of 0.80 mL/min, using a mobile phase of 20 mM Tris-HCl, 1.0 M sodium chloride, pH 8.00. Protein concentration was continuously determined during the run, by measuring light absorbance of the sample elution at the 280 nm wavelength. Exemplary results are shown in FIG. 6.

While certain compounds, compositions and methods described herein have been described with specificity in accordance with certain embodiments, the following examples serve only to illustrate the compounds of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

1. A method for large-scale production of recombinant iduronate-2-sulfatase (I2S) protein in mammalian cells, comprising culturing mammalian cells co-expressing a recombinant I2S protein and a formylglycine generating enzyme (FGE) in suspension in a large-scale culture vessel containing medium lacking serum.

2. The method of claim 1, wherein the cells, on average, produce the recombinant I2S protein at a specific productivity rate of greater than about 15 picogram/cell/day and further wherein the produced recombinant I2S protein, on average, comprises at least about 60% conversion of the cysteine residue corresponding to Cys59 of human I2S protein to C_{α}-formylglycine.

3. The method of claim 1, wherein the culturing step comprises a perfusion process.

4.-7. (canceled)

8. The method of claim 1, wherein the produced recombinant I2S protein, on average, comprises at least about 70% conversion of the cysteine residue corresponding to Cys59 of human I2S protein to C_{α}-formylglycine.

9.-10. (canceled)

11. The method of claim 1, wherein the mammalian cells are human cells.

12. The method of claim 1, wherein the mammalian cells are CHO cells.

13. The method of claim 1, wherein the large-scale culture vessel is a bioreactor.

14.-17. (canceled)

18. The method of claim 1, wherein the medium comprises at least one redox-modulator selected from the group consisting of glutathione, glucose-6-phosphate, carnosine, carnosol, sulforaphane, tocopherol, ascorbate, dehydroascorbate, selenium, 2-mercaptoenthanol, N-acetylcysteine, cysteine, riboflavin, niacin, folate, flavin adenine dinucleotide (FAD), and nicotinamide adenine dinucleotide phosphate (NADP).

19.-25. (canceled)

26. The method of claim 1, wherein the medium comprises at least one growth-modulator selected from the group consisting of hypoxanthine and thymidine.

27.-32. (canceled)

33. The method of claim 1, wherein the culturing step comprises a growth phase and a production phase.

34. The method of claim 33, wherein the mammalian cells are cultured at a temperature ranging from 30-37° C.

35. The method of claim 33, wherein the mammalian cells are cultured at different temperatures during the growth phase and the production phase.

36. The method of claim 33, wherein the medium for the growth phase and the production phase has different pH.

37. The method of any one of claim 33, wherein the mammalian cells are maintained at a viable cell density ranging from about 1.0-50×10⁶ viable cells/mL during the production phase.

38.-43. (canceled)

44. The method of claim 1, wherein the cells comprises one or more exogenous nucleic acids encoding the recombinant I2S protein and/or the FGE.

45.-47. (canceled)

48. The method of claim 1, wherein the cells over-express the FGE.

49. A recombinant iduronate-2-sulfatase (I2S) protein produced using the method of claim 1.

50. A preparation of recombinant iduronate-2-sulfatase (I2S) protein, said recombinant I2S protein having an amino acid sequence at least 70% identical to SEQ ID NO:1 and comprising at least about 70% conversion of the cysteine residue corresponding to Cys59 of SEQ ID NO:1 to C_{α}-formylglycine (FGly).

51.-54. (canceled)

55. The preparation of claim 50, wherein the recombinant I2S protein has specific activity of at least 40 U/mg as determined by an in vitro sulfate release activity assay using heparin disaccharide as substrate.

56.-59. (canceled)

60. A pharmaceutical composition comprising a recombinant I2S protein of claim 50 and a pharmaceutically acceptable carrier.

61. A method of treating Hunter syndrome comprising administering into a subject in need of treatment a pharmaceutical composition of claim 60.


SEQUENCE LISTING (6 sequences)

SEQ ID NO: 1 - protein, 525 aa, Homo sapiens
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 3 - protein, 312 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQEDQSSTGFRLKTSSTRKYK

SEQ ID NO: 4 - protein, 343 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGFLMRTNT

SEQ ID NO: 5 - protein, 341 aa, Homo sapiens
SQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 6 - protein, 374 aa, Homo sapiens
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD